SEQUENCE LISTING (60 sequences)

SEQ ID NO: 1 - DNA, 18 bp, Artificial Sequence
  primer 1
ggatcaggccatttttgg

SEQ ID NO: 2 - DNA, 18 bp, Artificial Sequence
  primer 2
ggatcatgccatttttgg

SEQ ID NO: 3 - DNA, 18 bp, Artificial Sequence
  primer 3
ggatcagtccatttttgg

SEQ ID NO: 4 - DNA, 18 bp, Artificial Sequence
  primer 4
ggatcaggacatttttgg

SEQ ID NO: 5 - DNA, 18 bp, Artificial Sequence
  primer 5
ggatcaggcaatttttgg

SEQ ID NO: 6 - DNA, 18 bp, Artificial Sequence
  primer 6
ggatcaggccttttttgg

SEQ ID NO: 7 - DNA, 18 bp, Artificial Sequence
  primer 7
gaagctgttgttatcact

SEQ ID NO: 8 - DNA, 67 bp, Artificial Sequence
  DNA template 1
ccccaaaaaggagcttgaggttctcctttaaaaagaagctgttgtattgttgtcgaagaagaaaagt

SEQ ID NO: 9 - DNA, 62 bp, Artificial Sequence
  DNA template 2
cccaaccctacccaccctcaagaaaaaaaagtgataattgttgtcgaagaagaaaaaaaatt

SEQ ID NO: 10 - RNA, 40 nt, Artificial Sequence
  target RNA 1
ggguuggccaaaggagaaccucaagcuccuggccugaucc

SEQ ID NO: 11 - RNA, 40 nt, Artificial Sequence
  target RNA 2
ggguuggccaaaggagaaccucaagcuccaggccugaucc

SEQ ID NO: 12 - RNA, 39 nt, Artificial Sequence
  no target RNA
ggccuggaggaggucuuuggcagguccccucucgggaaa

SEQ ID NO: 13 - DNA, 40 bp, Artificial Sequence
  target DNA 1
gggttggccaaaggagaacctcaagctcctggcctgatcc

SEQ ID NO: 14 - DNA, 40 bp, Artificial Sequence
  target DNA 2
gggttggccaaaggagaacctcaagctccaggcctgatcc

SEQ ID NO: 15 - DNA, 40 bp, Artificial Sequence
  Complementary DNA
ggatcaggccaggagcttgaggttctcctttggccaaccc

SEQ ID NO: 16 - DNA, 17 bp, Artificial Sequence
  primer 8
ggatcaggccatttttg

SEQ ID NO: 17 - DNA, 18 bp, Artificial Sequence
  primer 9
ggatcaggccatttttag

SEQ ID NO: 18 - DNA, 18 bp, Artificial Sequence
  primer 10
ggatcaggccattttttg

SEQ ID NO: 19 - DNA, 18 bp, Artificial Sequence
  primer 11
ggatcaggccatttttga

SEQ ID NO: 20 - DNA, 70 bp, Artificial Sequence
  DNA template 3
ccccaaaaaggagcttgaggttctcctttaaaggaaagaagctgttgtattgttgtcgaagaagaaaagt

SEQ ID NO: 21 - DNA, 73 bp, Artificial Sequence
  DNA template 4
ccccaaaaaggagcttgaggttctcctttaaaggagaaaagaagctgttgtattgttgtcgaagaagaaaagt

SEQ ID NO: 22 - DNA, 76 bp, Artificial Sequence
  DNA template 5
ccccaaaaaggagcttgaggttctcctttaaaggagaacctaagaagctgttgtattgttgtcgaagaagaaaagt

SEQ ID NO: 23 - DNA, 55 bp, Artificial Sequence
  DNA template 6
ccctaaaaaggagcttgaggttctcctttaaaaccttccccaccctccccaccct

SEQ ID NO: 24 - DNA, 55 bp, Artificial Sequence
  DNA template 7
cccaaaaaaggagcttgaggttctcctttaaaaccttccccaccctccccaccct

SEQ ID NO: 25 - DNA, 55 bp, Artificial Sequence
  DNA template 8
cctcaaaaaggagcttgaggttctcctttaaaaccttccccaccctccccaccct

SEQ ID NO: 26 - DNA, 55 bp, Artificial Sequence
  DNA template 9
ccccaaaaaggagcttgaggttctcctttaaaaccttccccaccctccccaccct

SEQ ID NO: 27 - DNA, 22 bp, Artificial Sequence
  oligonucleotide
agggttagggttagggttaggg

SEQ ID NO: 28 - DNA, 22 bp, Artificial Sequence
  oligonucleotide
agggagggcgctgggaggaggg

SEQ ID NO: 29 - DNA, 26 bp, Artificial Sequence
  oligonucleotide
ttagggttagggttagggttagggtt

SEQ ID NO: 30 - DNA, 27 bp, Artificial Sequence
  oligonucleotide
tggggagggtggggagggtggggaagg

SEQ ID NO: 31 - DNA, 20 bp, Artificial Sequence
  oligonucleotide
gggcggcgggctgggcgggg

SEQ ID NO: 32 - RNA, 18 nt, Artificial Sequence
  oligonucleotide
gggaggggcgggucuggg

SEQ ID NO: 33 - DNA, 18 bp, Artificial Sequence
  P1
gaagctgttgttatcact

SEQ ID NO: 34 - DNA, 18 bp, Artificial Sequence
  P2
ttacctggatcttttttg

SEQ ID NO: 35 - DNA, 18 bp, Artificial Sequence
  P3
ttacctggattttttttg

SEQ ID NO: 36 - DNA, 18 bp, Artificial Sequence
  P4
tatgggttcccttttttg

SEQ ID NO: 37 - DNA, 18 bp, Artificial Sequence
  P5
tatgggttcctttttttg

SEQ ID NO: 38 - DNA, 62 bp, Artificial Sequence; identical to SEQ ID NO: 9
  T1

SEQ ID NO: 39 - DNA, 73 bp, Artificial Sequence
  T2
caaaaaacagggggtgcttacaatcctaggattgtatagaagctgttgtattgttgtcgaagaagaaaagtcc

SEQ ID NO: 40 - DNA, 73 bp, Artificial Sequence
  T3
caaaaaagggaaataatcaatgatagtactatcatttagaagctgttgtattgttgtcgaagaagaaaagtcc

SEQ ID NO: 41 - DNA, 18 bp, Artificial Sequence
  P6
gaagctgttgttatcact

SEQ ID NO: 42 - DNA, 34 bp, Artificial Sequence
  Capture Probe 1
aaaggagaacctcaagctcctcagtctgataagc

SEQ ID NO: 43 - DNA, 35 bp, Artificial Sequence
  Capture Probe 2
aaaggagaacctcaagctccaaaatggctgtgata

SEQ ID NO: 44 - DNA, 67 bp, Artificial Sequence
  T4
ctgtcaacaggagcttgaggttctcctttaaaaagaagctgttgtattgttgtcgaagaagaaaagt

SEQ ID NO: 45 - DNA, 66 bp, Artificial Sequence
  T5
cactcatcggagcttgaggttctcctttaaaaagaagctgttgtattgttgtcgaagaagaaaagt

SEQ ID NO: 46 - RNA, 24 nt, Artificial Sequence
  miR-21CA
uagcuuaucagacugauguugaca

SEQ ID NO: 47 - RNA, 22 nt, Artificial Sequence
  miR-21
uagcuuaucagacugauguuga

SEQ ID NO: 48 - RNA, 22 nt, Artificial Sequence
  miR-13a
uaucacagccauuuugacgagu

SEQ ID NO: 49 - RNA, 22 nt, Artificial Sequence
  miR-13b
uaucacagccauuuugaugagu

SEQ ID NO: 50 - RNA, 23 nt, Artificial Sequence
  miR-221
agcuacauugucugcuggguuuc

SEQ ID NO: 51 - DNA, 30 bp, Artificial Sequence
  positive control primer
agctccttttttgggacttttcttcttcga

SEQ ID NO: 52 - DNA, 52 bp, Artificial Sequence
  DNA template t2
cccaaccctacccaccctcaagaaaaaaaagtgataattgttgtcgaagaag

SEQ ID NO: 53 - DNA, 40 bp, Artificial Sequence
  G636 sense
gaaaacatcaggattgtaagcaccccctggatccaggtaa

SEQ ID NO: 54 - DNA, 40 bp, Artificial Sequence
  G636 antisense
ttacctggatccagggggtgcttacaatcctgatgttttc

SEQ ID NO: 55 - DNA, 40 bp, Artificial Sequence
  G636A sense
gaaaacatcaggattgtaagcaccccctgaatccaggtaa

SEQ ID NO: 56 - DNA, 40 bp, Artificial Sequence
  G636A antisense
ttacctggattcagggggtgcttacaatcctgatgttttc

SEQ ID NO: 57 - DNA, 40 bp, Artificial Sequence
  G681 sense
aattttcccactatcattgattatttcccgggaacccata

SEQ ID NO: 58 - DNA, 40 bp, Artificial Sequence
  G681 antisense
tatgggttcccgggaaataatcaatgatagtgggaaaatt

SEQ ID NO: 59 - DNA, 40 bp, Artificial Sequence
  G681A sense
aattttcccactatcattgattatttcccaggaacccata

SEQ ID NO: 60 - DNA, 40 bp, Artificial Sequence
  G681A antisense
tatgggttcctgggaaataatcaatgatagtgggaaaatt

(19) United States
(12) Patent Application Publication
(10) Pub. No.: US 2021/0262015 A1
(43) Pub. Date: Aug. 26, 2021

(54) METHOD FOR DETECTING POLYNUCLEOTIDE SEQUENCE HAVING GENE MUTATION

(71) Applicant: National University Corporation Gunma University, Gunma (JP)

(72) Inventor: Masayasu Kuwahara, Gunma (JP)

(21) Appl. No.: 16/320,528

(22) PCT Filed: Jun. 1, 2017
(86) PCT No.: PCT/JP2017/020485

§ 371 Date: Jan. 25, 2019

(30) Foreign Application Priority Data
Jul. 25, 2016 (JP) 2016-145839
Mar. 15, 2017 (JP) 2017-050490

(51) Int. Cl. C12Q 1/6827

(52) U.S. Cl. CPC C12Q 1/6827; C12Q 2600/178; C12Q 2600/156

(57) ABSTRACT

A method for detecting a gene mutation(s), the method comprising the steps of: hybridizing a single-stranded circular DNA and a primer with a target polynucleotide containing a first region and a second region adjacent to the 3′-side of the first region and containing a mutation; performing a nucleic acid amplification reaction by rolling circle amplification based on formation of a complex of the target polynucleotide, the primer, and the single-stranded circular DNA; and detecting amplified nucleic acid with a detection reagent. In this method, the single-stranded circular DNA contains a sequence complementary to the first region of the target polynucleotide, a primer-binding sequence adjacent to the 5′-side thereof, and preferably a sequence complementary to a detection reagent-binding sequence. The oligonucleotide primer contains a region having a sequence complementary to the second region of the target polynucleotide, and a region adjacent to the 3′-side thereof and having a sequence complementary to the primer-binding sequence of the single-stranded circular DNA.

### TECHNICAL FIELD

The present invention relates to a method for simply and efficiently detecting a polynucleotide containing a gene mutation such as a single nucleotide polymorphism (SNP), base insertion, or base deletion.

### BACKGROUND ART

In recent years, a number of gene mutations, such as SNPs, associated with diseases have been discovered, and increased attention has been paid to genetic tests for analyzing genetic mutations for prediction of likelihood of developing diseases, and personalized medicine for determination of the therapeutic method depending on the types of gene mutations. For example, as methods for SNP analysis, a method in which a SNP-specific primer or a SNP-specific probe is used to investigate the type of a SNP based on whether amplification or hybridization occurs or not, a method in which the type of a SNP is investigated based on whether cleavage by a restriction enzyme occurs or not, a method in which sequencing is directly carried out, and the like have been known. However, expensive devices and high usage cost, as well as complicated operation prevent use of these methods for simple tests at clinics or for self-medication.

A method in which RNA is detected by the rolling circle amplification method has been disclosed in Patent Document 1. However, this method enables only detection of the sequence at the 3′-end since the method uses the analyte RNA as a primer. This method is insufficient also from the viewpoint of the amplification efficiency and the detection efficiency.

### PRIOR ART DOCUMENTS

### Patent Document

[Patent Document 1] JP 2012-080871 A

### Non-patent Document

[Non-patent Document 1] Anal. Chem., 2016, 88 (14), pp 7137-7144

### SUMMARY OF THE INVENTION

An object of the present invention is to provide a simple method for efficiently detecting gene mutations such as SNPs, and base insertions and base deletions, present at particular sites in target polynucleotides.

In order to solve the above problems, the present inventors intensively studied. As a result, the present inventors found that the method for detection of a polynucleotide that has already been filed as PCT/JP2016/059262 (WO 2016/152936), wherein a single-stranded circular DNA, a primer, and a guanine quadruplex-binding reagent are used (Non-patent Document 1), enables efficient identification of the type or the presence or absence of a gene mutation by using a target polynucleotide containing a gene mutation such as a SNP, designing a primer in a region containing the genetic polymorphism, and then investigating whether amplification occurs or not, thereby completing the present invention.

According to the present invention,

a method for detecting a gene mutation(s), the method comprising the steps of:

hybridizing a single-stranded circular DNA and an oligonucleotide primer with a capture polynucleotide containing: a first region; and a second region adjacent to the 3′-side of the first region;

performing a nucleic acid amplification reaction by rolling circle amplification based on formation of a complex of the capture polynucleotide, the primer, and the single-stranded circular DNA; and

detecting amplified nucleic acid, preferably a detection reagent-binding sequence contained in amplified nucleic acid, with a detection reagent;

wherein

the single-stranded circular DNA contains:
a sequence of 10 to 30 bases complementary to the first region of the capture polynucleotide;
a primer-binding sequence adjacent to the 5′-side thereof; and
preferably a sequence complementary to the detection reagent-binding sequence;

the oligonucleotide primer contains:
(ii) a region having a sequence of 8 to 15 bases complementary to the second region of the capture polynucleotide; and
a region adjacent to the 3′-side thereof and having a sequence complementary to the primer-binding sequence of the single-stranded circular DNA; and
the capture polynucleotide or the oligonucleotide primer contains a mutation, and mutation-specific hybridization of the capture polynucleotide, the primer, and the single-stranded circular DNA occurs to form the ternary complex to cause the nucleic acid amplification reaction, followed by the detection with the detection reagent;

is provided.

According to the first mode of the present invention, which is a method wherein a target polynucleotide containing a mutation is arranged in the position of the capture polynucleotide,

a method for detecting a gene mutation(s), the method comprising the steps of:

hybridizing a single-stranded circular DNA and a primer with a target polynucleotide containing: a first region; and a second region adjacent to the 3′-side of the first region and containing a mutation;

performing a nucleic acid amplification reaction by rolling circle amplification based on formation of a complex of the target polynucleotide, the primer, and the single-stranded circular DNA; and

detecting amplified nucleic acid, preferably a detection reagent-binding sequence contained in amplified nucleic acid, with a detection reagent;

wherein

the single-stranded circular DNA contains:
a sequence of 10 to 30 bases complementary to the first region of the target polynucleotide;
a primer-binding sequence adjacent to the 5′-side thereof; and
preferably a sequence complementary to the detection reagent-binding sequence; and

the oligonucleotide primer contains:
(ii) a region having a sequence of 8 to 15 bases complementary to the second region of the target polynucleotide; and
a region adjacent to the 3′-side thereof and having a sequence complementary to the primer-binding sequence of the single-stranded circular DNA;

is provided.

According to the second mode of the present invention, which is a method wherein a target polynucleotide containing a mutation is arranged in the position of the capture polynucleotide,

a method for detecting a gene mutation(s), the method comprising the steps of:

hybridizing a first single-stranded circular DNA and a first primer with a target polynucleotide containing: a first region; and a second region adjacent to the 3′-side of the first region and containing a mutation;

performing a nucleic acid amplification reaction by rolling circle amplification based on formation of a complex of the target polynucleotide, the first primer, and the first single-stranded circular DNA;

hybridizing a second single-stranded circular DNA and a second oligonucleotide primer with an extended chain generated by the nucleic acid amplification reaction, and performing a nucleic acid amplification reaction based on formation of a complex of the extended chain, the second primer, and the second single-stranded circular DNA; and

detecting amplified nucleic acid, preferably a detection reagent-binding sequence contained in amplified nucleic acid, with a detection reagent;

wherein

the first single-stranded circular DNA contains:
a sequence of 10 to 30 bases complementary to the first region of the target polynucleotide;
a primer-binding sequence adjacent to the 5′-side thereof; and
a sequence complementary to a second-single-stranded-circular-DNA-binding sequence;

the first oligonucleotide primer contains:
(ii) a region having a sequence of 8 to 15 bases complementary to the second region of the target polynucleotide; and
a region adjacent to the 3′-side thereof and having a sequence complementary to the primer-binding sequence of the first single-stranded circular DNA;
the second single-stranded circular DNA contains:
(iii) the same sequence as the sequence, in the first single-stranded circular DNA, complementary to the second-single-stranded-circular-DNA-binding sequence;
a second primer-binding sequence adjacent to the 5′-side thereof; and
preferably a sequence complementary to the detection reagent-binding sequence; and

the second oligonucleotide primer contains:
(iv) the same sequence as the region adjacent to the 5′-side of the sequence, in the first single-stranded circular DNA, complementary to the second-single-stranded-circular-DNA-binding sequence; and
a sequence adjacent to the 3′-side thereof and complementary to the second primer-binding sequence in the second single-stranded circular DNA;

is provided.

The oligonucleotide primer (the first oligonucleotide primer in the second mode) preferably has a base that hybridizes with a mutated base present in the second region of the target polynucleotide, which base in the primer is located at the 3′-end of the region having a sequence of 8 to 15 bases complementary to the second region of the target polynucleotide.

According to the third mode of the present invention, which is a method wherein a target miRNA (microRNA) containing a mutation is arranged in the position of the oligonucleotide,

a method for detecting a gene mutation(s), the method comprising the steps of:

hybridizing a single-stranded circular DNA and a capture polynucleotide with a miRNA containing: a first region; and a second region located in the 3′-side thereof and containing a mutation;

performing a nucleic acid amplification reaction by rolling circle amplification based on formation of a complex of the capture polynucleotide, the miRNA, and the single-stranded circular DNA; and

detecting amplified nucleic acid, preferably a detection reagent-binding sequence, with a detection reagent;

wherein

the single-stranded circular DNA contains:
a miRNA-binding region complementary to the second region of the miRNA;
a second region in the 3′-side thereof; and
preferably a sequence complementary to the detection reagent-binding sequence; and

the capture polynucleotide contains:
a sequence complementary to the second region of the single-stranded circular DNA; and
a miRNA-binding sequence complementary to the first region of the miRNA; is provided.

According to the fourth mode of the present invention, which is a method wherein a target miRNA (microRNA) containing a mutation is arranged in the position of the oligonucleotide,

a method for detecting a gene mutation(s), the method comprising the steps of:
hybridizing a first single-stranded circular DNA and a capture polynucleotide with a miRNA containing: a first region; and a second region adjacent to the 3′-side of the first region and containing a mutation;
performing a nucleic acid amplification reaction by rolling circle amplification based on formation of a complex of the miRNA, the capture polynucleotide, and the first single-stranded circular DNA;
hybridizing a second single-stranded circular DNA and a second oligonucleotide primer with an extended chain generated by the nucleic acid amplification reaction, and performing a nucleic acid amplification reaction based on formation of a complex of the extended chain, the second primer, and the second single-stranded circular DNA; and
detecting amplified nucleic acid, preferably a detection reagent-binding sequence contained in amplified nucleic acid, with a detection reagent;

wherein

the first single-stranded circular DNA contains:
a miRNA-binding region complementary to the second region of the miRNA;
a second region in the 3′-side thereof; and
a sequence complementary to a second-single-stranded-circular-DNA-binding sequence;
the capture polynucleotide contains:
a sequence complementary to the second region of the single-stranded circular DNA; and
a sequence complementary to the first region of the miRNA;

the second single-stranded circular DNA contains:
(iii) the same sequence as the sequence, in the first single-stranded circular DNA, complementary to the second-single-stranded-circular-DNA-binding sequence;
a second primer-binding sequence adjacent to the 5′-side thereof; and
preferably a sequence complementary to the detection reagent-binding sequence; and

the second oligonucleotide primer contains:
(iv) the same sequence as the region adjacent to the 5′-side of the sequence, in the first single-stranded circular DNA, complementary to the second-single-stranded-circular-DNA-binding sequence; and
a sequence adjacent to the 3′-side thereof and complementary to the second primer-binding sequence in the second single-stranded circular DNA;

is provided.

The detection reagent-binding sequence is preferably a guanine quadruplex-forming sequence, and the detection reagent is preferably a guanine quadruplex-binding reagent. The guanine quadruplex-binding reagent is preferably the later-mentioned ThT derivative.

According to one mode of the present invention, in the presence of a target polynucleotide sequence, a single-stranded circular DNA and a primer hybridize with the target polynucleotide sequence, and, from the resulting hybridization product, a DNA chain containing a number of detection reagent-binding sequences such as guanine quadruplex-containing sequences linearly bound to each other is produced. By staining the DNA chain with a detection reagent such as ThT (derivative), the target polynucleotide sequence can be specifically detected. Since the present invention uses the RCA method, in which the reaction proceeds at a constant temperature, rather than the PCR method, which requires a temperature cycle of, for example, increasing/decreasing the temperature, the present invention can be applied to simple detection methods. When there is a mismatch between a mutated base in the target polynucleotide sequence and a base in the primer, the amplification hardly occurs. Thus, identification of the type or the presence or absence of the mutation is possible, and therefore the present invention is useful for genetic testing and the like.

### BRIEF DESCRIPTION OF THE DRAWINGS

FIG. 1 shows a schematic diagram illustrating the polynucleotide amplification method according to the first mode of the present invention. Each star symbol indicates a SNP site.

FIG. 2 shows a schematic diagram illustrating the polynucleotide amplification method according to the second mode of the present invention. Each star symbol indicates a SNP site.

FIG. 3 shows a diagram illustrating formation of the complex of the target polynucleotide, the first single-stranded circular DNA, and each type of first primer, and formation of the complex of the resulting extension product, the second single-stranded circular DNA, and the second primer, in Example 1 (Samples b2 to b7).

FIG. 4 shows a diagram (photographs) showing the results of detection of the target polynucleotide in Example 1.

FIG. 5 shows a diagram illustrating formation of the complex of the target polynucleotide, the single-stranded circular DNA, and each type of primer, and formation of the complex of the resulting extension product, the second single-stranded circular DNA, and the second primer, in Example 2 (Samples a2, a4, b2, b4, b5, and b6).

FIG. 6 shows a diagram (photographs) showing the results of detection of the target polynucleotides in Example 2.

FIG. 7 shows a diagram showing the difference in the fluorescence spectrum obtained between the cases where a double-stranded DNA was targeted and the cases where a single-stranded DNA was targeted in Example 2.

FIG. 8 shows a diagram illustrating formation of the complex of the target polynucleotide, the single-stranded circular DNA, and each type of primer, and formation of the complex of the resulting extension product, the second single-stranded circular DNA, and the second primer, in Example 3 (Samples b2, b3, b4, b5, c2, c3, c4, and c5).

FIG. 9 shows a diagram (photographs) showing the results of detection of the target polynucleotide in Example 3.

FIG. 10 shows a diagram illustrating the structure of the complex of the target polynucleotide, the single-stranded circular DNA, and each type of primer in Example 4 (Samples b1, b2, b3, b4, and b5).

FIG. 11 shows a diagram (photographs) showing the results of detection of the target polynucleotide in Example 4.

FIG. 12 shows a diagram illustrating formation of the complex of the target polynucleotide, the single-stranded circular DNA, and each type of primer, and formation of the complex of the resulting extension product, the second single-stranded circular DNA, and the second primer, in Example 5 (left: G636 SNP, primer P2; right: G681 SNP, primer P4).

FIG. 13 shows a diagram (photographs) showing the results of detection of the target polynucleotides in Example 5.

FIG. 14 shows a schematic diagram illustrating the polynucleotide amplification method according to the third mode of the present invention. The star symbol indicates a SNP site.

FIG. 15 shows a schematic diagram illustrating the polynucleotide amplification method according to the fourth mode of the present invention. The star symbol indicates a SNP site.

FIG. 16 shows a diagram illustrating the structure of the complex of the target polynucleotide (miRNA), the single-stranded circular DNAs, the capture polynucleotide, and the primer in Example 6 (miR-21-CA).

FIG. 17 shows a diagram (photographs) showing the results of detection of the target polynucleotides in Example 6.

FIG. 18 shows a diagram illustrating the structure of the complex of the target polynucleotide (miRNA), the single-stranded circular DNAs, the capture polynucleotide, and the primer in Example 7 (miR-13(u)).

FIG. 19 shows a diagram (photographs) showing the results of detection of the target polynucleotides in Example 7.

FIG. 20 shows a diagram illustrating the structures of the complexes of the target polynucleotide (long-chain DNA), the single-stranded circular DNAs, the capture polynucleotide, and the primer in Example 8.

FIG. 21 shows a diagram (photographs) showing the results of detection of the target polynucleotide in Example 8 (use of a PCR amplification product).

FIG. 22 shows a diagram (photographs) showing the results of detection of the target polynucleotide in Example 8 (use of an extract).

### EMBODIMENTS FOR CARRYING OUT THE INVENTION

According to the present invention,

a method for detecting a gene mutation(s), the method comprising the steps of:
hybridizing a single-stranded circular DNA and an oligonucleotide primer with a capture polynucleotide containing: a first region; and a second region adjacent to the 3′-side of the first region;
performing a nucleic acid amplification reaction by rolling circle amplification based on formation of a complex of the capture polynucleotide, the primer, and the single-stranded circular DNA; and
detecting amplified nucleic acid, preferably a detection reagent-binding sequence contained in amplified nucleic acid, with a detection reagent;

wherein

the single-stranded circular DNA contains:
a sequence of 10 to 30 bases complementary to the first region of the capture polynucleotide;
a primer-binding sequence adjacent to the 5′-side thereof; and
preferably a sequence complementary to the detection reagent-binding sequence; the oligonucleotide primer contains:
a region having a sequence of 8 to 15 bases complementary to the second region of the capture polynucleotide; and
a region adjacent to the 3′-side thereof and having a sequence complementary to the primer-binding sequence of the single-stranded circular DNA; and
the capture polynucleotide or the oligonucleotide primer contains a mutation, and mutation-specific hybridization of the capture polynucleotide, the primer, and the single-stranded circular DNA occurs to form the ternary complex to cause the nucleic acid amplification reaction, followed by the detection with the detection reagent;

is provided.

Examples of the gene mutation herein include single nucleotide polymorphisms (SNPs), base deletions, and base insertions.

### <Use of Capture Polynucleotide as Target Polynucleotide Containing Mutation: First Mode>

A method for detecting a gene mutation(s) according to the first mode of the present invention uses

a target polynucleotide containing: a first region; and a second region adjacent to the 3′-side thereof and containing a mutation;

and uses

(i) a single-stranded circular DNA containing:
a sequence of 10 to 30 bases complementary to the first region of the target polynucleotide;
a primer-binding sequence adjacent to the 5′-side thereof; and
a sequence complementary to a detection reagent-binding sequence;

(ii) an oligonucleotide primer containing:
a sequence of 8 to 15 bases complementary to the second region, in the target polynucleotide, adjacent to the 3′-side of the first region and containing a mutation; and
a sequence adjacent to the 3′-side thereof and complementary to the primer-binding sequence of the single-stranded circular DNA; and

(iii) a detection reagent.

### <Target Polynucleotide>

The target polynucleotide is not limited as long as it is a sequence containing a gene mutation such as a SNP. The target polynucleotide may be either DNA or RNA. The DNA may be either a single-stranded DNA, or a double-stranded DNA composed of a sense strand and an antisense strand (complementary strand).

Regarding the target polynucleotide, for example, in genomic DNA or RNA, a region containing a target gene mutation may be set as the second region; the region adjacent to the 5′-side thereof may be set as the first region; and a sequence containing these may be set as the target polynucleotide. Thereafter, based on the sequences of the first region and the second region, the following single-stranded circular DNA and primer may be designed.

The target polynucleotide may be prepared or isolated from a sample derived from a biological species. As a sample containing such a target polynucleotide, the individual itself of a virus, prokaryote, or eukaryote, or a part thereof may be used. In cases of vertebrates (including human), examples of the sample include excrements such as feces, urine, and sweat; and body fluids such as blood, semen, saliva, gastric juice, and bile. The sample may also be a tissue surgically removed from a body, or a tissue dropped from a body such as a body hair. The sample may also be a prepared polynucleotide-containing product that is prepared from a processed product of food or the like. The sample may also be an RNA-containing product prepared by further fractionating the above sample and then separating a part thereof. As long as the sample contains the target polynucleotide, the polynucleotide may be either purified or unpurified.

The length of the target polynucleotide is not limited as long as the hybridization with the single-stranded circular DNA and the primer is possible. Since hybridization of the single-stranded circular DNA easily occurs with a loop portion of a stem-loop structure, the target polynucleotide preferably has a stem-loop structure.

### <Single-stranded Circular DNA>

The single-stranded circular DNA contains:

a sequence of 10 to 30 bases complementary to the first region of the target polynucleotide;

a primer-binding sequence of preferably 7 to 8 bases adjacent to the 5′-side thereof; and

a sequence complementary to a detection reagent-binding sequence such as a guanine quadruplex-forming sequence.

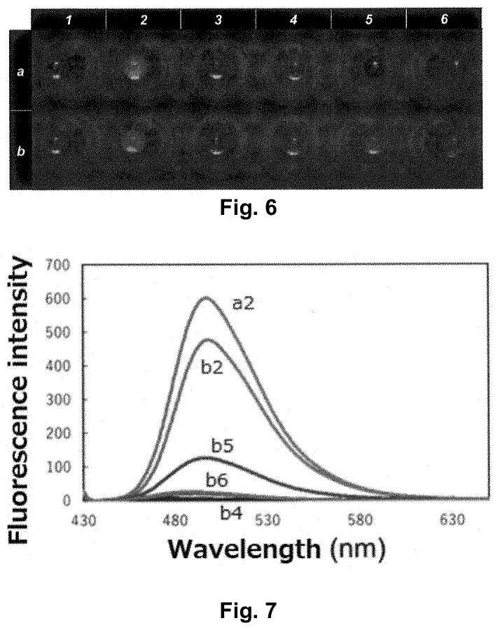

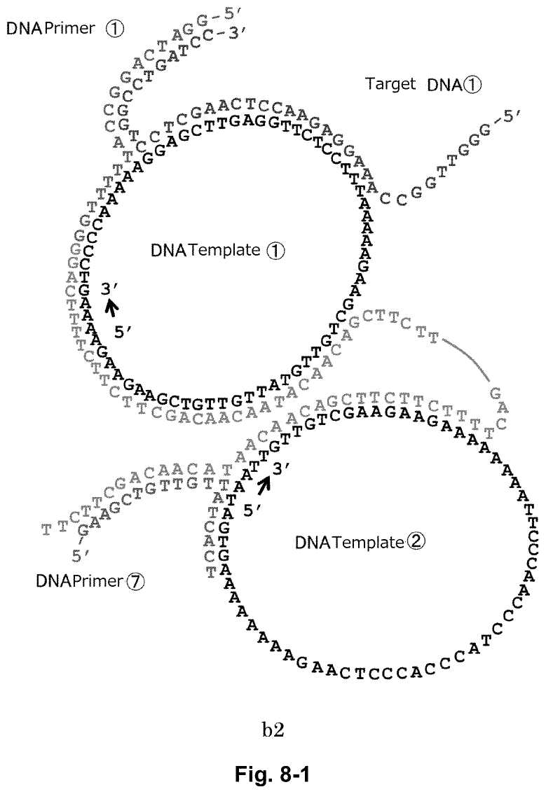

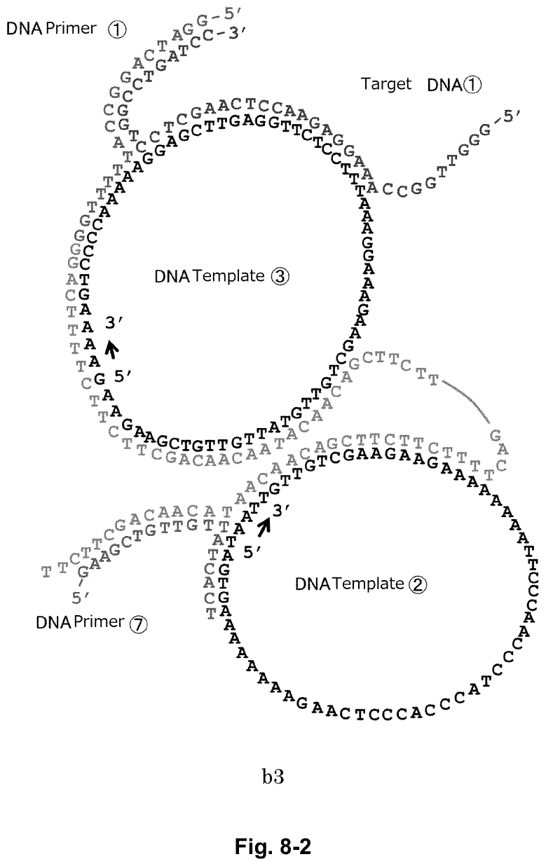

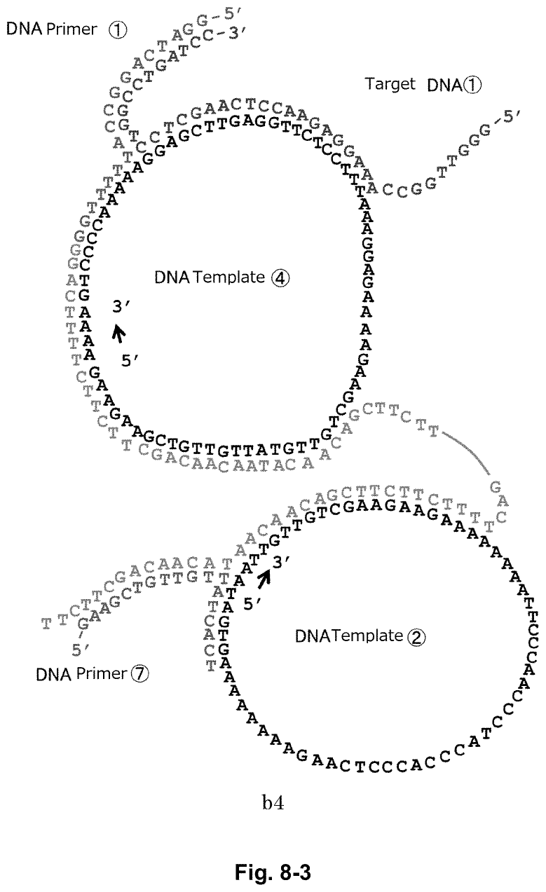

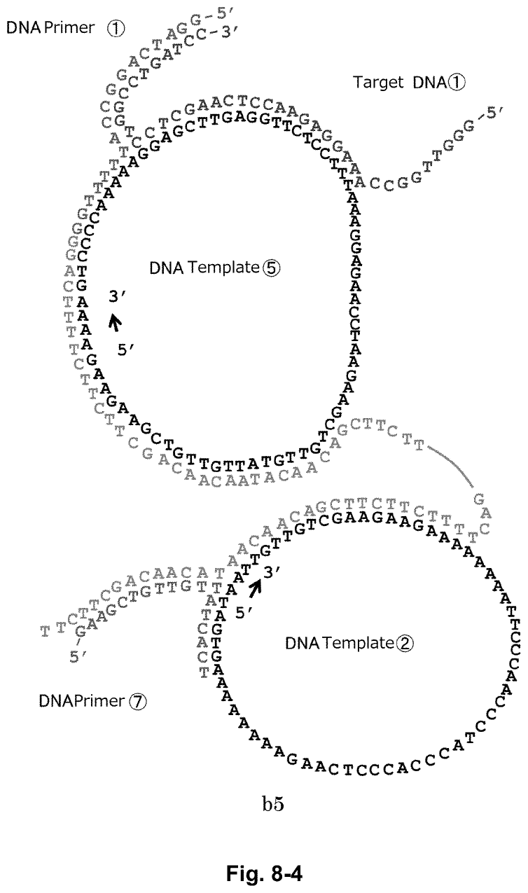

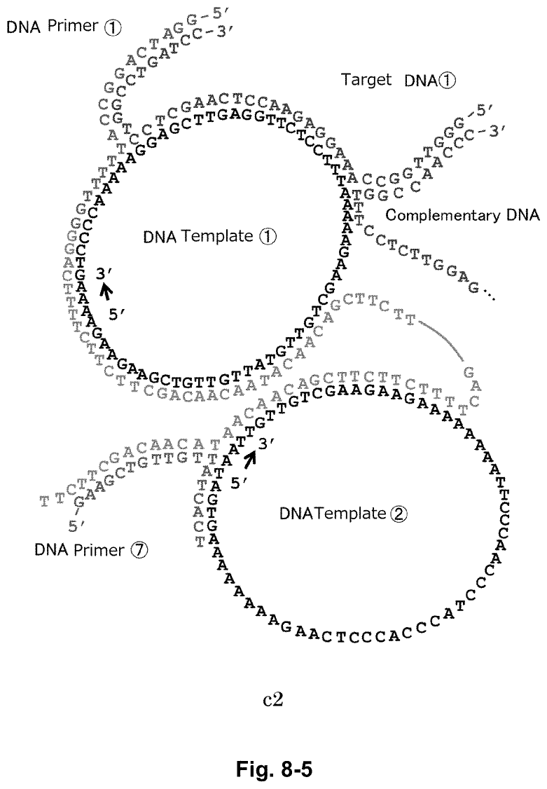

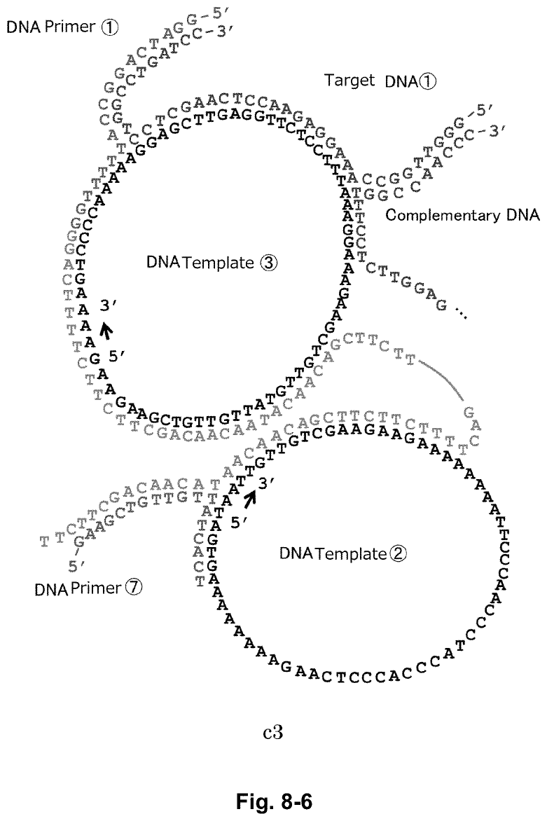

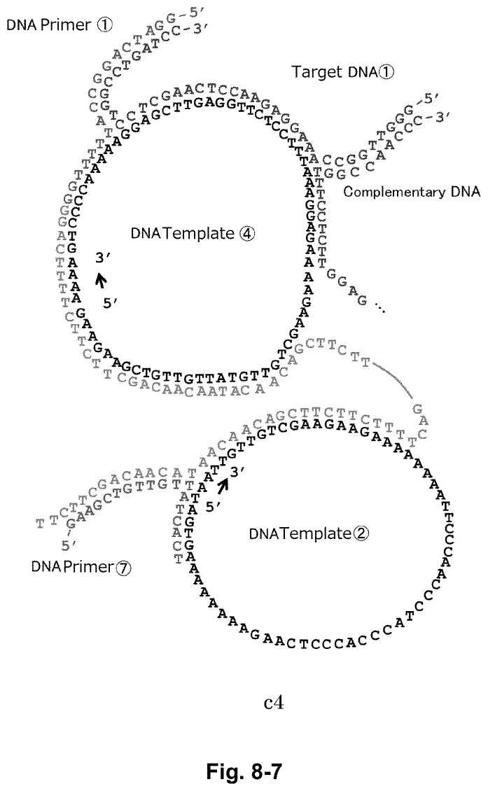

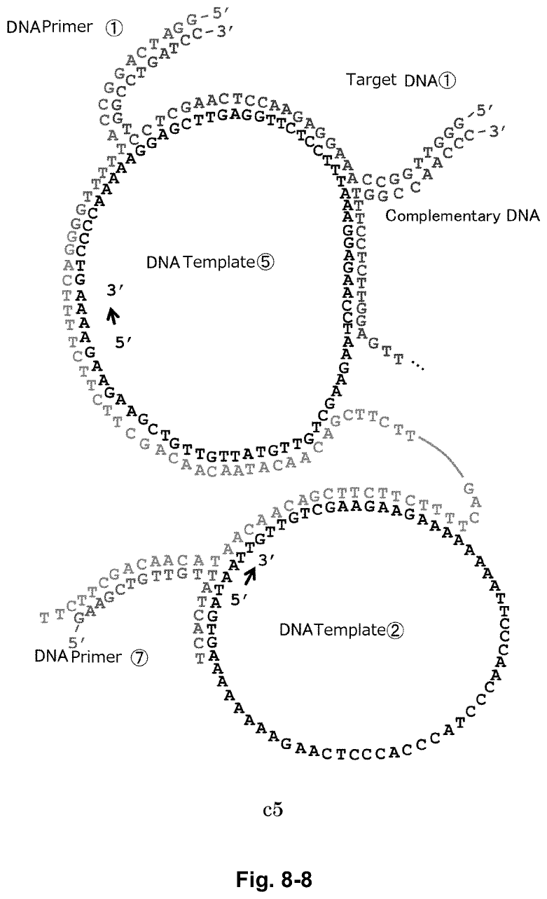

In cases where the target polynucleotide is a double-stranded DNA, as shown in c2, c3, and c5 in FIG. 8, the single-stranded circular DNA preferably further contains a sequence which is adjacent to the 3′-side of the sequence of 10 to 30 bases complementary to the first region of the target polynucleotide, and which is complementary to the 3′-end side in this sequence. By this, hybridization of the complementary strand of the double-stranded DNA is possible, and the complex can be more stable.

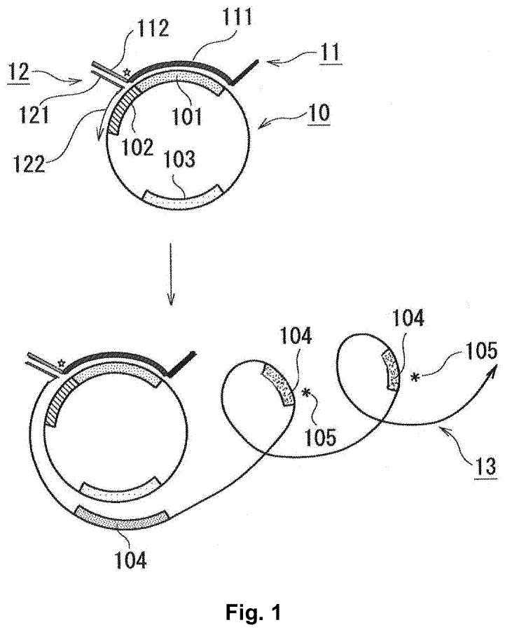

A description is given below showing an example with reference to FIG. 1. The single-stranded circular DNA is illustrated in the 5′→3′ clockwise direction. A single-stranded circular DNA 10 contains: a sequence 101 complementary to a first region 111 of a target polynucleotide 11; a primer-binding sequence 102 linked to the 5′-side thereof; and a sequence 103 complementary to a guanine quadruplex-forming sequence.

The sequence 101 has a length of usually 10 to 30 bases, preferably 15 to 25 bases, and a GC content of preferably 30 to 70%. The sequence 102 has a length of preferably 7 bases or 8 bases. The sequence is not limited, and has a GC content of preferably 30 to 70%. Examples of the guanine quadruplex-forming sequence include a sequence described in Nat Rev Drug Discov. 2011 Apr; 10(4): 261-275, and can be represented as G₃N₁₋₁₀G₃N₁₋₁₀G₃N₁₋₁₀G₃. Specific examples of the sequence include the sequences of SEQ ID NOs:27 to 32. Examples of the sequence complementary to the guanine quadruplex-forming sequence include C₃N₁₋₁₀C₃N₁₋₁₀C₃N₁₋₁₀C₃. That is, in the sequence, three consecutive C′s are repeated four times via spacers each having a sequence composed of one to ten (preferably one to five) arbitrary bases (N=A, T, G, or C).

22mer DNA (22AG: 5′-AGGGTTAGGGTTAGGGTTAGGG-3′
(SEQ ID NO: 27)
and
22Kit: 5′-AGGGAGGGCGCTGGGAGGAGGG-3′ (SEQ ID
NO: 28)),
26mer DNA (26Tel: 5′-TTAGGGTTAGGGTTAGGGTTAGGGTT-3′
(SEQ ID NO: 29)),
27mer DNA (27Myc: 5′-TGGGGAGGGTGGGGAGGGTGGGGA
AGG-3′ (SEQ ID NO: 30)),
20mer DNA (20Src: 5′-GGGCGGCGGGCTGGGCGGGG-3′ (SEQ
ID NO: 31)),
18mer RNA (18Ras: 5′-GGGAGGGGCGGGUCUGGG-3′ (SEQ
ID NO: 32)).

The sequence complementary to the guanine quadruplex-forming sequence may have arbitrary sequences before and after it, that is, between it and the primer-binding sequence 102, and between it and the sequence 101 complementary to the first region of the target polynucleotide. The total length of the single-stranded circular DNA 10 is preferably 35 to 100 bases.

Although FIG. 1 describes a case where the detection reagent-binding sequence is a guanine quadruplex-forming sequence, the detection may also be carried out using, for example, as the detection reagent-binding sequence, an aptamer sequence or a molecular beacon (hairpin-shaped oligonucleotide having a fluorescent group (donor) and a quenching group (acceptor) that cause FRET)-binding sequence, and using, as the detection reagent, an aptamer-binding coloring molecule or a molecular beacon. The detection may also be carried out using a labeled probe that hybridizes with the detection reagent-binding sequence.

In the first mode of the present invention, it is also possible to detect the amplified nucleic acid using, as the detection reagent, a nucleic acid staining reagent which non-sequence-specifically binds to DNA to emit fluorescence, such as Cyber Gold (trade name). Therefore, in the single-stranded circular DNA, the presence of the sequence complementary to the detection reagent-binding sequence is not indispensable.

The single-stranded circular DNA 10 can be obtained by circularization of a single-stranded DNA (ssDNA). The circularization of the single-stranded DNA can be carried out by arbitrary means. It can be carried out by using, for example, CircLigase (registered trademark), CircLigase II (registered trademark), ssDNA Ligase (Epicentre), or ThermoPhage ligase (registered trademark) single-stranded DNA (Prokzyme).

### <Oligonucleotide Primer>

A primer 12 contains:

a target polynucleotide-binding sequence 121 of 8 to 15 bases complementary to a second region 112 of the target polynucleotide 11, which second region 112 is adjacent to the 3′-side of the first region 111 and contains a gene mutation such as a SNP (the star symbols in FIG. 1); and

a circular DNA-binding sequence 122 of preferably 7 to 8 bases linked to the 3′-side thereof and complementary to the primer-binding region 102 of the single-stranded circular DNA 10.

The primer may be provided as an immobilized primer by, for example, immobilization on a carrier. By this, detection on the solid phase becomes possible. Examples of the method of the immobilization include a method in which the primer is labeled with biotin or the like, and then immobilized by interaction with avidin or the like.

The oligonucleotide primer preferably has a base that hybridizes with the mutated base present in the second region of the target polynucleotide, which base in the primer is preferably located at the 3′-end of the sequence 121 complementary to the second region of the target polynucleotide.

For example, in a case where the target polynucleotide has an A/G single nucleotide polymorphism, and where the A is to be detected, the oligonucleotide primer is especially preferably designed such that T, corresponding to the A, is located at the 3′-end of the target polynucleotide-binding sequence.

### <Amplification Method>

After hybridizing the single-stranded circular DNA 10 and the primer 12 with the target polynucleotide 11 to allow formation of a complex of these three molecules, nucleic acid amplification reaction based on the target polynucleotide is carried out using the rolling circle amplification (RCA) method.

Those skilled in the art can appropriately set the conditions for the hybridization taking into account the combination of the single-stranded circular DNA, the target polynucleotide, and the primer.

The RCA method is described in, for example, Lizardi et al., Nature Genet. 19: 225-232 (1998); U.S. Pat. No. 5,854,033 B; U.S. Pat. No. 6,143,495 B; and WO 97/19193. The RCA method can be carried out using a mesophilic chain-substituting DNA synthetase such as phi29 polymerase, Klenow DNA Polymerase (5′-3′, 3′-5′ exo minus), Sequenase (registered trademark) Version 2.0 T7 DNA Polymerase (USB), Bsu DNA Polymerase, or Large Fragment (NEB); or a heat-resistant chain-substituting DNA synthetase such as Bst DNA Polymerase (Large Fragment), Bsm DNA Polymerase, Large Fragment (Fermentas), BcaBEST DNA polymerase (TakaraBio), Vent DNA polymerase (NEB), Deep Vent DNA polymerase (NEB), or DisplaceAce (registered trademark) DNA Polymerase (Epicentre).

The extension reaction of DNA by RCA does not require use of a thermal cycler, and is carried out, for example, at a constant temperature within the range of 25° C. to 65° C. The reaction temperature is appropriately set according to a normal procedure based on the optimum temperature of the enzyme and the denaturation temperature (the temperature range in which binding (annealing) of the primer to, or dissociation of the primer from, the template DNA occurs), which is dependent on the primer chain length. The reaction may also be carried out at a constant, relatively low temperature. For example, in cases where phi29DNA polymerase is used as the chain-substituting DNA synthetase, the reaction is carried out preferably at 25° C. to 42° C., more preferably at about 30 to 37° C. By the RCA, nucleic acid (amplification product 13) containing a guanine quadruplex-forming sequence (corresponding to the sequence 103) is amplified dependently on the target polynucleotide 11 from the primer 12 along the single-stranded circular DNA 10. Since the amplification product 13 contains a sequence 104 containing a guanine quadruplex, it can be detected with a guanine quadruplex detection reagent 105.

In cases where the type of the gene mutation such as a SNP in the target polynucleotide matches the type of the base arranged in the primer, that is, in cases where the mutant base in the target polynucleotide corresponds to the base which is contained in the primer and complementary to the base at the mutation site, amplification reaction occurs, so that the amplification product can be detected with the detection reagent. On the other hand, in cases where the type of the mutation in the target polynucleotide is different from the type of the base arranged in the primer, amplification reaction hardly occurs, so that the amplification product cannot be detected with the detection reagent. Thus, by the detection method of the present invention, the type of the gene mutation such as a SNP, or the presence or absence of the gene mutation, in the target polynucleotide can be identified.

The first and second modes described above are designed such that a mutation is contained in the second region of the target polynucleotide, and extension reaction from the primer is analyzed based on stability of the complex, wherein the stability is based on hybridization with the primer, which has the base corresponding to the mutation. However, the method of the present invention also include a mode designed such that a mutation is contained in the first region of the target polynucleotide, and extension reaction from the primer is analyzed based on stability of the complex, wherein the stability is based on hybridization with the single-stranded circular DNA, which has the base corresponding to the mutation.

### <Use of Capture Polynucleotide as Target Polynucleotide Containing Mutation: Second Mode>

A method for detecting a gene mutation(s) according to the second mode of the present invention uses

a target polynucleotide containing: a first region; and a second region adjacent to the 3′-side thereof and containing a mutation;

and uses

(i) a first single-stranded circular DNA containing:
a sequence of 10 to 30 bases complementary to the first region of the target polynucleotide;
a first primer-binding sequence adjacent to the 5′-side thereof; and
a sequence complementary to a second-single-stranded-circular-DNA-binding sequence;

(ii) a first oligonucleotide primer containing:
a sequence of 8 to 15 bases complementary to the second region, in the target polynucleotide, adjacent to the 3′-side of the first region and containing a mutation; and
a sequence adjacent to the 3′-side thereof and complementary to the first primer-binding region of the first single-stranded circular DNA;

(iii) a second single-stranded circular DNA containing:
the same sequence as the sequence, in the first single-stranded circular DNA, complementary to the second-single-stranded-circular-DNA-binding sequence;
a second primer-binding sequence adjacent to the 5′-side thereof; and
a sequence complementary to a detection reagent-binding sequence such as a guanine quadruplex-forming sequence;

(iv) a second oligonucleotide primer containing:
the same sequence as the region adjacent to the 5′-side of the sequence, in the first single-stranded circular DNA, complementary to the second-single-stranded-circular-DNA-binding sequence; and
a sequence adjacent to the 3′-side thereof and complementary to the second primer-binding sequence in the second single-stranded circular DNA; and

(v) a detection reagent such a guanine quadruplex-binding reagent.

### <Target Polynucleotide>

The target polynucleotide is as described for the first mode.

### <First Single-stranded Circular DNA>

The first single-stranded circular DNA contains:

a sequence of 10 to 30 bases complementary to the first region of the target polynucleotide;

a primer-binding sequence of preferably 7 to 8 bases adjacent to the 5′-side thereof; and

a sequence complementary to a second-single-stranded-circular-DNA-binding sequence.

In cases where the target polynucleotide is a double-stranded DNA, as shown in c2, c3, and c5 in FIG. 8, the first single-stranded circular DNA preferably further contains a sequence which is adjacent to the 3′-side of the sequence of 10 to 30 bases complementary to the first region of the target polynucleotide, and which is complementary to the 3′-end side in this sequence. By this, hybridization of the complementary strand of the double-stranded DNA is possible, and the complex can be more stable.

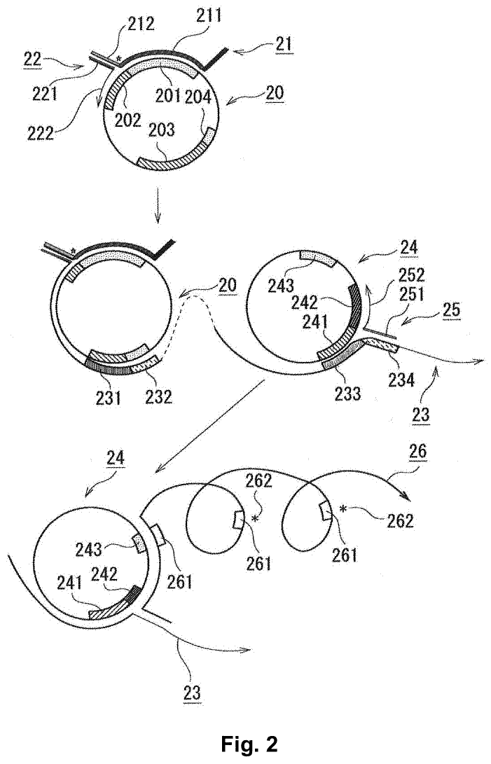

A description is given below with reference to FIG. 2.

A first single-stranded circular DNA 20 contains:

a sequence 201 complementary to a first region 211 of a target polynucleotide 21;

a primer-binding sequence 202 linked to the 5′-side thereof; and

a sequence 203 complementary to a second-single-stranded-circular-DNA-binding sequence.

The sequence 201 has a length of usually 10 to 30 bases, preferably 15 to 25 bases, and a GC content of preferably 30 to 70%. The sequence 202 has a length of 7 bases or 8 bases. The sequence is not limited, and has a GC content of preferably 30 to 70%. The total length of the first single-stranded circular DNA 20 is preferably 35 to 100 bases. The first single-stranded circular DNA 20 can be obtained by circularization of a single-stranded DNA (ssDNA) by the method described above.

### <First Oligonucleotide Primer>

A first oligonucleotide primer 22 contains:

a sequence 221 of 8 to 15 bases complementary to a second region 212 of the target polynucleotide 21, which second region 212 is adjacent to the 3′-side of the first region 211 and contains a gene mutation; and

a sequence 222 of preferably 7 to 8 bases linked to the 3′-side thereof and complementary to the primer-binding region 202 of the first single-stranded circular DNA 20.

The first oligonucleotide primer preferably has a base that hybridizes with the mutated base present in the second region of the target polynucleotide, which base in the primer is preferably located at the 3′-end of the sequence 221 complementary to the second region of the target polynucleotide.

For example, in a case where the gene mutation in the target polynucleotide is an A/G single nucleotide polymorphism, and where the A is to be detected, the first oligonucleotide primer is preferably designed such that T, corresponding to the A, is located at the 3′-end of the target polynucleotide-binding sequence 221.

### <Second Single-stranded Circular DNA>

A second single-stranded circular DNA 24 contains:

the same sequence 241 as the sequence 203, in the first single-stranded circular DNA 20, complementary to the second-single-stranded-circular-DNA-binding sequence;

a second primer-binding sequence 242 adjacent to the 5′-side thereof; and

a sequence 243 complementary to a guanine quadruplex-forming sequence.

The sequence 241 has a length of usually 10 to 30 bases, preferably 15 to 25 bases, and a GC content of preferably 30 to 70%. The sequence 242 has a length of 7 bases or 8 bases. The sequence is not limited, and has a GC content of preferably 30 to 70%. The sequence 243 complementary to a guanine quadruplex-forming sequence is the same as that described for the first mode. The total length of the second single-stranded circular DNA 24 is preferably 35 to 100 bases. The second single-stranded circular DNA 24 can be obtained by circularization of a single-stranded DNA (ssDNA) by the method described above.

Although FIG. 2 describes a case where the detection reagent-binding sequence is a guanine quadruplex-forming sequence, the detection may also be carried out using, as the detection reagent-binding sequence, an aptamer sequence or a molecular beacon (hairpin-shaped oligonucleotide having a fluorescent group (donor) and a quenching group (acceptor) that cause FRET)-binding sequence, and using, as the detection reagent, an aptamer-binding coloring molecule or a molecular beacon (ChemBioChem 2007, 8, 1795-1803; J. Am. Chem. Soc. 2013, 135, 7430-7433).

In the second mode of the present invention, it is also possible to detect the amplified nucleic acid using, as the detection reagent, a nucleic acid staining reagent which non-sequence-specifically binds to DNA to emit fluorescence, such as Cyber Gold (trade name). Therefore, in the second single-stranded circular DNA, the presence of the sequence complementary to the detection reagent-binding sequence is not indispensable.

### <Second Oligonucleotide Primer>

A second oligonucleotide primer 25 contains:

the same sequence 251 (preferably a sequence of 8 to 15 bases) as a region 204, in the first single-stranded circular DNA 20, adjacent to the 5′-side of the sequence 203 complementary to the second-single-stranded-circular-DNA-binding sequence; and

a sequence 252 (preferably a sequence of 7 to 8 bases) adjacent to the 3′-side thereof and complementary to the second primer-binding sequence 242 of the second single-stranded circular DNA.

### <Amplification Method>

As shown in FIG. 2, after hybridizing the first single-stranded circular DNA 20 and the primer 22 with the target polynucleotide 21 to allow formation of a complex of these three molecules, nucleic acid amplification reaction based on the target polynucleotide is carried out using the rolling circle amplification (RCA) method. The reaction conditions and the like are the same as those for the first mode.

By the RCA, a first amplification product 23 is amplified dependently on the target polynucleotide 21 from the primer 22 along the first single-stranded circular DNA 20.

The amplification product 23 contains a sequence 231 complementary to the sequence 203, in the first single-stranded circular DNA 20, complementary to the second-single-stranded-circular-DNA-binding sequence. Therefore, the second single-stranded circular DNA 24, which contains the same sequence 241 as the sequence 203, hybridizes with the sequence 231 of the first amplification product 23 via the sequence 241.

With the thus formed complex of the first amplification product 23 and the second single-stranded circular DNA, the second oligonucleotide primer 25 hybridizes to form a complex of these three molecules.

That is, since the second oligonucleotide primer 25 contains the same sequence 251 as the region 204, in the first single-stranded circular DNA 20, adjacent to the 5′-side of the sequence 203 complementary to the second-single-stranded-circular-DNA-binding sequence, the second oligonucleotide primer 25 hybridizes with the region 232 of the first amplification product 23, which region is complementary to the region 204 of the first single-stranded circular DNA 20, via the sequence 251.

Since the second oligonucleotide primer 25 contains, in the 3′-side of the sequence 251, the sequence 252 complementary to the second primer-binding sequence 242 of the second single-stranded circular DNA 24, the second oligonucleotide primer 25 also hybridizes with the second single-stranded circular DNA 24 via the sequence 252.

By RCA, a second amplification product 26 (extended chain) is amplified from the resulting ternary complex of the first amplification product 23, the second single-stranded circular DNA 24, and the second oligonucleotide primer 25. Since the second amplification product 26 contains a sequence 261 containing a guanine quadruplex, it can be detected with a guanine quadruplex detection reagent 262. In the second mode, the second single-stranded circular DNA 24 hybridizes with each region 231 contained in the first amplification product 23 to cause the RCA reaction. Thus, a remarkable improvement in the detection sensitivity can be achieved.

In cases where the type of the gene mutation such as a SNP in the target polynucleotide matches the type of the base arranged in the primer, amplification reaction occurs, so that the amplification product can be detected with the detection reagent. On the other hand, in cases where the type of the gene mutation in the target polynucleotide is different from the type of the base arranged in the primer, amplification reaction hardly occurs, so that the amplification product cannot be detected with the detection reagent.

Thus, by the detection method of the present invention, the type of the gene mutation such as a SNP, or the presence or absence of the gene mutation, in the target polynucleotide can be identified.

### <Use of Oligonucleotide Primer as Target Polynucleotide Containing Mutation: Third Mode>

A method for detecting a gene mutation(s) according to the third mode of the present invention uses, as a target polynucleotide,

a miRNA containing: a first region; and a second region in the 3′-side thereof containing a mutation;

and uses, for performing a reaction,

(i) a single-stranded circular DNA containing:
a miRNA-binding region complementary to the second region of the miRNA;
a second region in the 3′-side thereof; and
a sequence complementary to a detection reagent-binding sequence such as a guanine quadruplex;

(ii) a capture polynucleotide containing:
a template-binding sequence complementary to the second region of the single-stranded circular DNA; and
a miRNA-binding sequence complementary to the first region of the miRNA; and

(iii) a detection reagent.

### <Target Polynucleotide>

The target polynucleotide is a miRNA containing a gene mutation such as a SNP. As the miRNA, a miRNA having a mutation in its 3′-side, that is, a miRNA containing: a first region; and a second region in the 3′-side thereof containing a mutation; is used. The mutation is preferably present at the 3′-end of the miRNA, or at a position within 1 to 3 bases from the 3′-end. The miRNA has a length of preferably 15 to 30 bases, more preferably 15 to 25 bases, still more preferably 15 to 23 bases.

### <Single-stranded Circular DNA>

The single-stranded circular DNA contains:

a miRNA-binding region complementary to the second region of the miRNA;

a second region in the 3′-side thereof; and

a sequence complementary to a detection reagent-binding sequence such as a guanine quadruplex-forming sequence.

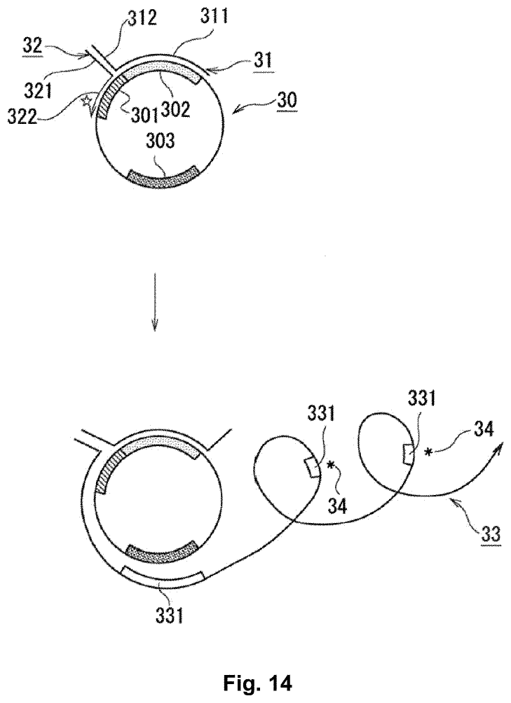

A description is given below showing an example with reference to FIG. 14. The single-stranded circular DNA is illustrated in the 5′→3′ clockwise direction. A single-stranded circular DNA 30 contains:

a sequence (miRNA-binding region) 301 complementary to a second region 322 of a target miRNA 32;

a second region 302 linked to the 3′-side thereof; and

a sequence 303 complementary to a guanine quadruplex-forming sequence.

The sequence 301 has a length of preferably 7 bases or 8 bases. The sequence is not limited, and has a GC content of preferably 30 to 70%. The second region 302 has a length of usually 10 to 30 bases, preferably 15 to 25 bases, and a GC content of preferably 30 to 70%.

The miRNA-binding region 301 in the single-stranded circular DNA 30, which region is complementary to the second region 322 of the miRNA 32, contains a base complementary to a mutation in the miRNA (the mutation to be detected: the star symbol in FIG. 14). The miRNA-binding region 301 does not hybridize when the mutation in the miRNA is different from the mutation to be detected. Accordingly, the presence or absence of the mutation of interest can be detected based on whether the hybridization occurs or not.

The sequence 303 complementary to the guanine quadruplex-forming sequence may have arbitrary sequences before and after it, that is, between it and the second region 302, and between it and the miRNA-binding region 301. The total length of the single-stranded circular DNA 30 is preferably 35 to 100 bases. The guanine quadruplex-forming sequence may be replaced with another detection reagent-binding sequence. In the third mode of the present invention, it is also possible to detect the amplified nucleic acid using, as the detection reagent, a nucleic acid staining reagent which non-sequence-specifically binds to DNA to emit fluorescence, such as Cyber Gold (trade name). Therefore, in the single-stranded circular DNA, the presence of the sequence complementary to the detection reagent-binding sequence is not indispensable.

### <Capture Polynucleotide>

The capture polynucleotide contains:

a template-binding sequence complementary to the second region of the single-stranded circular DNA; and

a miRNA-binding sequence complementary to the first region of the miRNA.

In FIG. 14, a capture polynucleotide 31 contains:

a sequence 311 complementary to the second region 302 of the single-stranded circular DNA 30; and

a miRNA-binding sequence 312 complementary to the first region 321 of the miRNA 32.

The sequence 311 has a length of usually 10 to 30 bases, preferably 15 to 25 bases, and a GC content of preferably 30 to 70%. The sequence 312 has a length of usually 8 to 15 bases, and a GC content of preferably 30 to 70%. In order to avoid non-specific amplification, the 3′-end of the capture polynucleotide is preferably modified with a phosphate group or the like.

### <Amplification Method>

After hybridizing the single-stranded circular DNA 30 and the capture polynucleotide 31 with the target polynucleotide (miRNA) 32 to allow formation of a complex of these three molecules, nucleic acid amplification reaction based on the target polynucleotide is carried out using the rolling circle amplification (RCA) method.

Those skilled in the art can appropriately set the conditions for the hybridization taking into account the combination of the single-stranded circular DNA, the target polynucleotide, and the primer.

By the RCA, an amplification product 33 is amplified from the target polynucleotide (miRNA) 32 along the single-stranded circular DNA 30. Since the amplification product 33 contains a sequence 331 containing a guanine quadruplex, it can be detected with a guanine quadruplex detection reagent 34.

In cases where the type of the miRNA mutation in the target polynucleotide matches the type of the base arranged in the miRNA-binding region of the single-stranded circular DNA, that is, in cases where the mutant base in the target polynucleotide corresponds to the base, contained in the single-stranded circular DNA, complementary to the base at the mutation site, amplification reaction occurs, so that the amplification product can be detected with the detection reagent. On the other hand, in cases where the type of the mutation in the target polynucleotide is different from the type of the base arranged in the single-stranded circular DNA, amplification reaction hardly occurs, so that the amplification product cannot be detected with the detection reagent. Thus, by the detection method of the present invention, the type of the gene mutation such as a SNP, or the presence or absence of the gene mutation, in the target polynucleotide can be identified.

### <Use of Oligonucleotide Primer as Target Polynucleotide Containing Mutation: Fourth Mode>

A method for detecting a gene mutation(s) according to the fourth mode of the present invention uses, as a target polynucleotide,

a miRNA containing: a first region; and a second region in the 3′-side thereof containing a mutation;

and uses

(i) a first single-stranded circular DNA containing:
a miRNA-binding region complementary to the second region of the miRNA;
a second region in the 3′-side thereof; and
a sequence complementary to a second-single-stranded-circular-DNA-binding sequence;

(ii) a capture polynucleotide containing:
a template-binding sequence complementary to the second region of the single-stranded circular DNA; and
a miRNA-binding sequence complementary to the first region of the miRNA;

(iii) a second single-stranded circular DNA containing:
the same sequence as the sequence, in the first single-stranded circular DNA, complementary to the second-single-stranded-circular-DNA-binding sequence;
a second primer-binding sequence adjacent to the 5′-side thereof; and
a sequence complementary to a detection reagent-binding sequence; and

(iv) a second oligonucleotide primer containing:
the same sequence as the region adjacent to the 5′-side of the sequence, in the first single-stranded circular DNA, complementary to the second-single-stranded-circular-DNA-binding sequence; and
a sequence adjacent to the 3′-side thereof and complementary to the second primer-binding sequence in the second single-stranded circular DNA.

<Target Polynucleotide>

The target polynucleotide and the capture polynucleotide are as described for the third mode.

### <First Single-stranded Circular DNA>

The single-stranded circular DNA contains:

a miRNA-binding region complementary to the second region of the miRNA;

a second region in the 3′-side thereof; and

a sequence complementary to a second-single-stranded-circular-DNA-binding sequence.

Since the miRNA-binding region complementary to the second region of the miRNA contains a sequence complementary to the mutation site in the miRNA, the presence or absence of the mutation can be detected based on whether hybridization occurs or not.

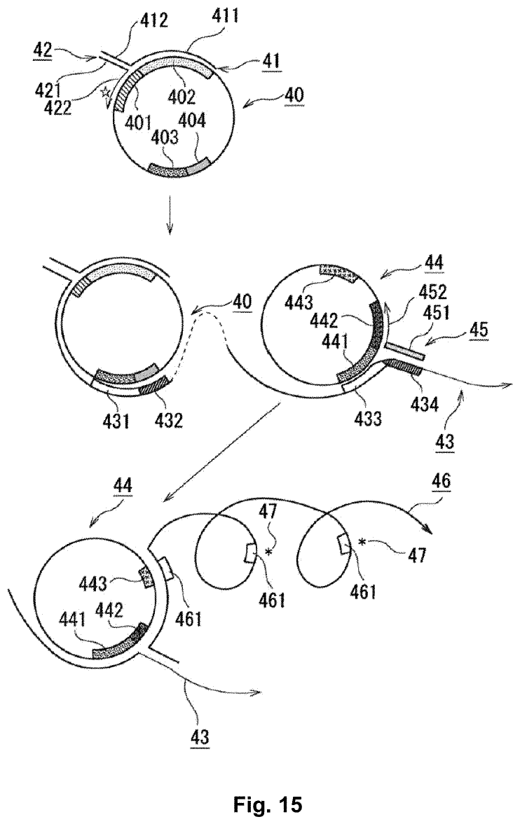

A description is given below showing an example with reference to FIG. 15. The single-stranded circular DNA is illustrated in the 5′→3′ clockwise direction. A single-stranded circular DNA 40 contains:

a sequence (miRNA-binding region) 401 complementary to a second region 422 of a target miRNA 42;

a second region 402 linked to the 3′-side thereof; and

a sequence 403 complementary to a second-single-stranded-circular-DNA-binding sequence.

The sequence 401 has a length of preferably 7 bases or 8 bases. The sequence is not limited, and has a GC content of preferably 30 to 70%. The sequence 402 has a length of usually 10 to 30 bases, preferably 15 to 25 bases, and a GC content of preferably 30 to 70%. The total length of the first single-stranded circular DNA 40 is preferably 35 to 100 bases. The first single-stranded circular DNA 40 can be obtained by circularization of a single-stranded DNA (ssDNA) by the method described above.

### <Second Single-stranded Circular DNA>

A second single-stranded circular DNA 44 contains:

the same sequence 441 as the sequence 403, in the first single-stranded circular DNA 40, complementary to the second-single-stranded-circular-DNA-binding sequence;

a second primer-binding sequence 442 adjacent to the 5′-side thereof; and

a sequence 443 complementary to a guanine quadruplex-forming sequence.

The sequence 441 has a length of usually 10 to 30 bases, preferably 15 to 25 bases, and a GC content of preferably 30 to 70%. The sequence 442 has a length of preferably 7 bases or 8 bases. The sequence is not limited, and has a GC content of preferably 30 to 70%. The sequence 443 complementary to a guanine quadruplex-forming sequence is the same as that described for the first mode. The total length of the second single-stranded circular DNA 44 is preferably 35 to 100 bases. The second single-stranded circular DNA 44 can be obtained by circularization of a single-stranded DNA (ssDNA) by the method described above.

Although FIG. 15 describes a case where the detection reagent-binding sequence is a guanine quadruplex-forming sequence, the detection may also be carried out using, as the detection reagent-binding sequence, an aptamer sequence or a molecular beacon (hairpin-shaped oligonucleotide having a fluorescent group (donor) and a quenching group (acceptor) that cause FRET)-binding sequence, and using, as the detection reagent, an aptamer-binding coloring molecule or a molecular beacon (ChemBioChem 2007, 8, 1795-1803; J. Am. Chem. Soc. 2013, 135, 7430-7433). As described above, in the fourth mode of the present invention, it is also possible to detect the amplified nucleic acid using, as the detection reagent, a nucleic acid staining reagent which non-sequence-specifically binds to DNA to emit fluorescence, such as Cyber Gold (trade name). Therefore, in the second single-stranded circular DNA, the presence of the sequence complementary to the detection reagent-binding sequence is not indispensable.

### <Second Oligonucleotide Primer>

A second oligonucleotide primer 45 contains:

the same sequence 451 (preferably a sequence of 8 to 15 bases) as a region 404, in the first single-stranded circular DNA 40, adjacent to the 5′-side of the sequence 403 complementary to the second-single-stranded-circular-DNA-binding sequence; and

a sequence 452 (preferably a sequence of 7 to 8 bases) adjacent to the 3′-side thereof and complementary to the second primer-binding sequence 442 of the second single-stranded circular DNA.

### <Amplification Method>

As shown in FIG. 15, after hybridizing the capture oligonucleotide 41 and the first single-stranded circular DNA 40 with the target polynucleotide (miRNA) 42 to allow formation of a complex of these three molecules, nucleic acid amplification reaction based on the target polynucleotide is carried out using the rolling circle amplification (RCA) method. The reaction conditions and the like are the same as those for the first mode.

By the RCA, a first amplification product 43 is amplified dependently on the target polynucleotide (miRNA) 42 along the first single-stranded circular DNA 40.

The amplification product 43 contains a sequence 431 complementary to the sequence 403, in the first single-stranded circular DNA 40, complementary to the second-single-stranded-circular-DNA-binding sequence. Therefore, the second single-stranded circular DNA 44, which contains the same sequence 441 as the sequence 403, hybridizes with the sequence 431 of the first amplification product 43 via the sequence 441.

With the thus formed complex of the first amplification product 43 and the second single-stranded circular DNA, the second oligonucleotide primer 45 hybridizes to form a complex of these three molecules.

That is, since the second oligonucleotide primer 45 contains the same sequence 451 as the region 404, in the first single-stranded circular DNA 40, adjacent to the 5′-side of the sequence 403 complementary to the second-single-stranded-circular-DNA-binding sequence, the second oligonucleotide primer 45 hybridizes with the region 432 of the first amplification product 43, which region is complementary to the region 404 of the first single-stranded circular DNA 40, via the sequence 451.

Since the second oligonucleotide primer 45 contains, in the 3′-side of the sequence 451, the sequence 452 complementary to the second primer-binding sequence 442 of the second single-stranded circular DNA 44, the second oligonucleotide primer 45 also hybridizes with the second single-stranded circular DNA 44 via the sequence 452.

By RCA, a second amplification product 46 (extended chain) is amplified from the resulting ternary complex of the first amplification product 43, the second single-stranded circular DNA 44, and the second oligonucleotide primer 45. Since the second amplification product 46 contains a sequence 461 containing a guanine quadruplex, it can be detected with a guanine quadruplex detection reagent 462. In the fourth mode, the second single-stranded circular DNA 44 hybridizes with each region 431 contained in the first amplification product 43 to cause the RCA reaction. Thus, a remarkable improvement in the detection sensitivity can be achieved.

In cases where the type of the gene mutation such as a SNP in the target polynucleotide matches the type of the base arranged in the primer, amplification reaction occurs, so that the amplification product can be detected with the detection reagent. On the other hand, in cases where the type of the gene mutation in the target polynucleotide is different from the type of the base arranged in the primer, amplification reaction hardly occurs, so that the amplification product cannot be detected with the detection reagent.

Thus, by the detection method of the present invention, the type of the gene mutation such as a SNP, or the presence or absence of the gene mutation, in the target polynucleotide can be identified.

### <Detection Reagent>

In the method of the present invention, a nucleic acid staining reagent which non-sequence-specifically binds to DNA to emit fluorescence, such as Cyber Gold (trade name), may be used as the detection reagent. However, for specific and highly sensitive detection, it is preferred to use a molecule which binds to a particular nucleic acid sequence (detection reagent-binding sequence) to cause luminescence or coloring.

As described above, the combination of the detection reagent-binding sequence and the detection reagent may be arbitrarily decided, and examples of the combination include combinations of an aptamer sequence and an aptamer-binding coloring molecule, combinations of a molecular beacon-binding molecule and a molecular beacon, and combinations of a specific sequence and a labeled probe that hybridizes therewith. The combination is preferably a combination of a guanine quadruplex and a guanine quadruplex-binding reagent. Examples of the guanine quadruplex-binding reagent include the following reagents.

[1] Thioflavin T (ThT) or a derivative thereof

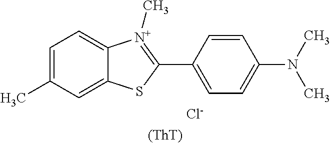

[2] H-aggregate “Yan, J. W.; Ye, W. J.; Chen, S. B.; Wu, W. B.; Hou, J. Q.; Ou, T. M.; Tan, J. H.; Li, D.; Gu, L. Q.; Huang, Z. S. Anal. Chem. 2012, 84, 6288-6292.”

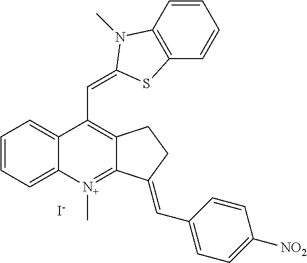 [SMILES: CN1/C(=C/c2c3c([n+](C)c4ccccc24)/C(=C/c2ccc([N+](=O)[O-])cc2)CC3)Sc2ccccc21 ; [I-]]

[3] TMPyP4 “Yaku, H.; Fujimoto, T.; Murashima, T.; Miyoshi, D.; Sugimoto, N. Chem. Commun. 2012, 48, 6203-6216.”

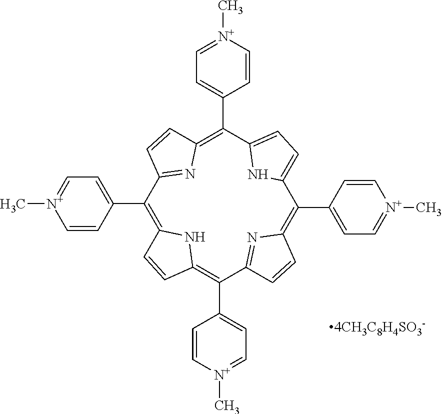

[4] PPIX “Li, T.; Wang, E.; Dong, S. Anal. Chem. 2010, 82, 7576-7580.”

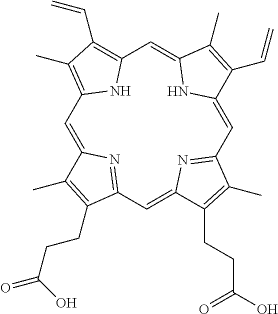 [SMILES: C=Cc1c(C)c2cc3[nH]c(cc4nc(cc5nc(cc1[nH]2)C(C)=C5CCC(=O)O)C(CCC(=O)O)=C4C)c(C)c3C=C]

[5] BPBC “Jin, B.; Zhang, X.; Zheng, W.; Liu, X.; Qi, C.; Wang, F.; Shangguan, D. Anal. Chem. 2014, 86, 943-952.”

[6] APD “Nikan, M.; Di Antonio, M.; Abecassis, K.; McLuckie, K.; Balasubramanian, S. Angew. Chem., Int. Ed. 2013, 52, 1428-1431.”

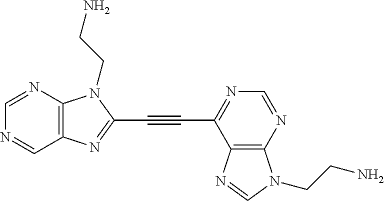 [SMILES: NCCn1cnc2c(C#Cc3nc4cncnc4n3CCN)ncnc21]

[7] Thiazole Orange (TO)

“Nakayama S.; Kelsey I.; Wang J.; Roelofs K.; Stefane B.; Luo Y.; Lee V.T.; Sintim H. 0. J. Am. Chem. Soc. 2011, 133, 4856-4864.”

Preferably, a ThT derivative represented by the following General Formula (I) may be used (Anal. Chem. 2014, 86, 12078-12084). JP 2016-079132 A.

In this formula, R¹ represents hydrogen, or a C₁-C₁₀ (preferably C₁-C₅) hydrocarbon group which optionally contains one or more selected from the group consisting of O, S, and N. The hydrocarbon group may be either linear or branched, or either saturated or unsaturated. The hydrocarbon group may be an aliphatic hydrocarbon group such as an alkyl group, or may be an aromatic hydrocarbon group such as an aryl group or an arylalkyl group. The term “optionally contains one or more selected from the group consisting of O, S, and N” means that the hydrocarbon group may contain a functional group containing a nitrogen atom, oxygen atom, sulfur atom, or the like, such as an amino group (—NR₂) (wherein each R independently represents hydrogen or a C₁-C₅ alkyl group), nitro group (—NO₂), cyano group (—CN), isocyanate group (—NCO), hydroxyl group (—OH), aldehyde group (—CHO), carboxyl group (—COOH), mercapto group (—SH), or sulfonic acid group (—SO₃H), or that a linking group containing a nitrogen atom, oxygen atom, sulfur atom, or the like, such as an ether group (—O—), imino group (—NH—), thioether group (—S—), carbonyl group (—C(═O)—), amide group (—C(═O)—NH—), ester group —C(═O)—O—), or thioester group (—C(═O)—S—), may be contained in the inside or at a terminus of the carbon backbone of the hydrocarbon group.

R², R³, and R⁴ each independently represent a C₁-C₅ (aliphatic) hydrocarbon group, more preferably a C₁-C₃ hydrocarbon group, especially preferably a methyl group. The C₁-C₅ hydrocarbon group may be either linear or branched, or either saturated or unsaturated.

n represents an integer of 0 to 5, more preferably an integer of 0 to 3, especially preferably 1.

X represents O, S, or NH, more preferably O.

Specific examples of the compound include the following.

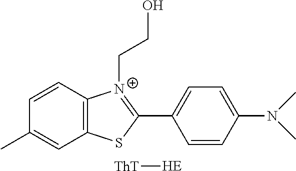

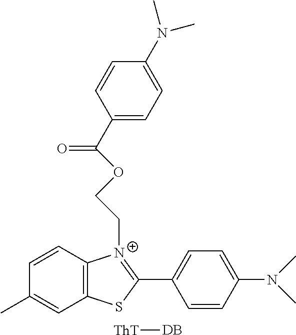

The detection of the guanine quadruplex structure in the test DNA can be carried out by, for example, bringing a compound represented by General Formula (I) or a salt thereof into contact with a sample containing the RCA product, and detecting the compound bound to the guanine quadruplex structure based on fluorescence emitted from the compound. The detection operation itself is the same as a known method except that the compound represented by General Formula (I) or a salt thereof is used. The detection operation can be carried out by bringing a solution prepared by dissolving the compound in a buffer into contact with a sample containing a test DNA, incubating the resulting mixture, carrying out washing, and then detecting fluorescence from the fluorescent dye bound to the test DNA after the washing.

### EXAMPLES

The present invention is described below by way of Examples. However, the present invention is not limited to the modes of the following Examples.

TABLE 1
Sequences of Oligonucleotides

| | Length | |
|---|---|---|
| | (mer) | Sequence (5′→3′) |
| DNA Primer (1) | 18 | GGATCAGGCCATTTTTGG (SEQ ID NO: 1) |
| DNA Primer (2) | 18 | GGATCATGCCATTTTTGG (SEQ ID NO: 2) |
| DNA Primer (3) | 18 | GGATCAGTCCATTTTTGG (SEQ ID NO: 3) |
| DNA Primer (4) | 18 | GGATCAGGACATTTTTGG (SEQ ID NO: 4) |
| DNA Primer (5) | 18 | GGATCAGGCAATTTTTGG (SEQ ID NO: 5) |
| DNA Primer (6) | 18 | GGATCAGGCCTTTTTTGG (SEQ ID NO: 6) |
| DNA Primer (7) | 18 | GAAGCTGTTGTTATCACT (SEQ ID NO: 7) |
| DNA Primer (8) | 17 | GGATCAGGCCATTTTTG (SEQ ID NO: 16) |
| DNA Primer (9) | 18 | GGATCAGGCCATTTTTAG (SEQ ID NO: 17) |
| DNA Primer (10) | 18 | GGATCAGGCCATTTTTTG (SEQ ID NO: 18) |
| DNA Primer (11) | 18 | GGATCAGGCCATTTTTGA (SEQ ID NO: 19) |
| DNA Template | 67 | CCCCAAAAAGGAGCTTGAGGTTCTCCTTTAAAAAGAAGCTGTTGTATTGTTG |
| (1) | | TCGAAGAAGAAAAGT (SEQ ID NO: 8) |
| DNA Template | 62 | CCCAACCCTACCCACCCTCAAGAAAAAAAAGTGATAATTGTTGTCGAAGAAG |
| (2) | | AAAAAAAATT (SEQ ID NO: 9) |
| DNA Template | 70 | CCCCAAAAAGGAGCTTGAGGTTCTCCTTTAAAGGAAAGAAGCTGTTGTATTG |
| (3) | | TTGTCGAAGAAGAAAAGT (SEQ ID NO: 20) |
| DNA Template | 73 | CCCCAAAAAGGAGCTTGAGGTTCTCCTTTAAAGGAGAAAAGAAGCTGTTGTA |
| (4) | | TTGTTGTCGAAGAAGAAAAGT (SEQ ID NO: 21) |
| DNA Template | 76 | CCCCAAAAAGGAGCTTGAGGTTCTCCTTTAAAGGAGAACCTAAGAAGCTGTT |
| (5) | | GTATTGTTGTCGAAGAAGAAAAGT (SEQ ID NO: 22) |
| DNA Template | 55 | CCCTAAAAAGGAGCTTGAGGTTCTCCTTTAAAACCTTCCCCACCCTCCCCACCCT |
| (6) | | (SEQ ID NO: 23) |
| DNA Template | 55 | CCCAAAAAAGGAGCTTGAGGTTCTCCTTTAAAACCTTCCCCACCCTCCCCACCCT |
| (7) | | (SEQ ID NO: 24) |
| DNA Template | 55 | CCTCAAAAAGGAGCTTGAGGTTCTCCITTAAAACCITCCCCACCCTCCCCACCCT |
| (8) | | (SEQ ID NO: 25) |
| DNA Template | 55 | CCCCAAAAAGGAGCTTGAGGTTCTCCTTTAAAACCTTCCCCACCCTCCCCACCCT |
| (9) | | (SEQ ID NO: 26) |
| Target RNA (1) | 40 | GGGUUGGCCAAAGGAGAACCUCAAGCUCCUGGCCUGAUCC (SEQ ID NO: 10) |
| Target RNA (2) | 40 | GGGUUGGCCAAAGGAGAACCUCAAGCUCCAGGCCUGAUCC (SEQ ID NO: 11) |
| no Target RNA | 39 | GGCCUGGAGGAGGUCUUUGGCAGGUCCCCUCUCGGGAAA (SEQ ID NO: 12) |
| Target DNA (1) | 40 | GGGTTGGCCAAAGGAGAACCTCAAGCTCCTGGCCTGATCC (SEQ ID NO: 13) |
| Target DNA (2) | 40 | GGGTTGGCCAAAGGAGAACCTCAAGCTCCAGGCCTGATCC (SEQ ID NO: 14) |
| Complementary DNA | 40 | GGATCAGGCCAGGAGCTTGAGGTTCTCCTTTGGCCAACCC (SEQ ID NO: 15) |

DNA primer (2) has the same sequence as the sequence of DNA primer (1) except that the G at base position 7 (7G) is changed to T.

DNA primer (3) has the same sequence as the sequence of DNA primer (1) except that the G at base position 8 (8G) is changed to T.

DNA primer (4) has the same sequence as the sequence of DNA primer (1) except that the C at base position 9 (9C) is changed to A.

DNA primer (5) has the same sequence as the sequence of DNA primer (1) except that the C at base position 10 (10C) is changed to A.

DNA primer (6) has the same sequence as the sequence of DNA primer (1) except that the A at base position 11 (11A) is changed to T.

DNA templates (1) to (9) are circularized.

Target RNA (2) has the same sequence as the sequence of target RNA (1) except that the U at base position 30 (30U) is changed to A.

Target DNA (2) has the same sequence as the sequence of target DNA (1) except that the T at base position 30 (30T) is changed to A.

Complementary DNA has the sequence complementary to target DNA (1).

[1] Circularization of DNA Template

(1) A mixture was prepared with 20 μL of 5 μM single-stranded DNA (Cid Pre T: 67mer; Cid Mai T: 62mer) (final concentration, 0.5 μM), 20 μL of 10 x attached buffer, 10 μL of 50 mM MnCl₂ (final concentration, 2.5 mM), 40 μL of 5 M betaine (final concentration, 1 M), 10 μL of 100 U/μL CircLigase (Epicentre Technologies, WI, USA) (final concentration, 5 U/μL), and 100 μL of water (200 μL in total).

(2) The mixture was incubated at 60° C. for 24 hours.

(3) PAGE purification was carried out. [2] Preparation of Reaction Solutions

### [Example 1] One-base Mismatch of Primer

### Preparation of a1 to a7 (Table 2)

A mixture was prepared with 2 μL of 100 nM DNA template (1) (final concentration, 10 nM), 2 μL of 400 nM DNA template (2) (final concentration, 40 nM), 2 μL of water or one of 120 nM DNA primers (1) to (6) (see Table 2) (final concentration, 12 nM), 2 μL of 480 nM DNA primer (7) (final concentration, 48 nM), 2 μL of 10×attached buffer, 2 μL of 10×attached BSA solution, 2 μL of 10 mM dNTPs (final concentration, 1 mM), 2 μL of 1 U/μL Phi29 Polymerase (New England Biolabs) (final concentration, 0.1 U/μL), and 4 μL of water (20 μL in total).

### Preparation of b1 to b7 (Table 2)

A mixture was prepared with 2 μL of 100 nM DNA template (1) (final concentration, 10 nM), 2 μL of 400 nM DNA template (2) (final concentration, 40 nM), 2 μL of water or one of 120 nM DNA primers (1) to (6) (see Table 2) (final concentration, 12 nM), 2 μL of 480 nM DNA primer (7) (final concentration, 48 nM), 2 μL of 10 x attached buffer, 2 μL of 10 x attached BSA solution, 2 μL of 10 mM dNTPs (final concentration, 1 mM), 2 μL of 1 U/μL Phi29 Polymerase (final concentration, 0.1 U/μL), 2 μL of 10 nM target RNA (1) (final concentration, 1 nM), and 2 μL of water (20 μL in total).

### Preparation of c1 to c7 (Table 2)

A mixture was prepared with 2 μL of 100 nM DNA template (1) (final concentration, 10 nM), 2 μL of 400 nM DNA template (2) (final concentration, 40 nM), 2 μL of water or one of 120 nM DNA primers (1) to (6) (see Table 2) (final concentration, 12 nM), 2 μL of 480 nM DNA primer (7) (final concentration, 48 nM), 2 μL of 10×attached buffer, 2 μL of 10 x attached BSA solution, 2 μL of 10 mM dNTPs (final concentration, 1 mM), 2 μL of 1 U/μL Phi29 Polymerase (final concentration, 0.1 U/μL), 2 μL of 10 nM no target RNA (final concentration, 1 nM), and 2 μL of water (20 μL in total).

### [Example 2] Difference between DNA/RNA Targets, and One-base Mismatch

### Preparation of al to a6 (Table 3)

A mixture was prepared with 2 μL of 100 nM DNA template (1) (final concentration, 10 nM), 2 μL of 400 nM DNA template (2) (final concentration, 40 nM), 2 μL of 120 nM DNA primer (1) (final concentration, 12 nM), 2 μL of 480 nM DNA primer (7) (final concentration, 48 nM), 2 μL of 10 x attached buffer, 2 μL of 10 x attached BSA solution, 2 μL of 10 mM dNTPs (final concentration, 1 mM), 2 μL of 1 U/μL Phi29 Polymerase (final concentration, 0.1 U/μL), 2 μL of water or 10 nM RNA (see Table 3), and 2 μL of water (20 μL in total).

### Preparation of b1 to b6, and a2, b1, b2, b4 to b6 (Tables 3 and 4)

A mixture was prepared with 10 μL of 100 nM DNA template (1) (final concentration, 10 nM), 10 μL of 400 nM DNA template (2) (final concentration, 40 nM), 10 μL of 120 nM DNA primer (1) (final concentration, 12 nM), 10 μL of 480 nM DNA primer (7) (final concentration, 48 nM), 10 μL of 10 x attached buffer, 10 μL of 10 x attached BSA solution, 10 μL of 10 mM dNTPs (final concentration, 1 mM), 10 μL of 1 U/μL Phi29 Polymerase (final concentration, 0.1 U/μL), 10 μL of water or 10 nM DNA (see Tables 3 and 4), and 10 μL of water (100 μL in total).

The double-stranded DNAs for b5 and b6 were subjected to denaturing and annealing in advance to form the double strands.

### [Example 3] Optimization of Circular Templates for Double-stranded DNA Target

### Preparation of a1 to a5 (Table 5)

A mixture was prepared with 2 μL of one of 100 nM DNA templates (1), (3), (4), and (5) (see Table 5) (final concentration, 10 nM), 2 μL of 400 nM DNA template (2) (final concentration, 40 nM), 2 μL of 120 nM DNA primer (1) (final concentration, 12 nM), 2 μL of 480 nM DNA primer (7) (final concentration, 48 nM), 2 μL of 10×attached buffer, 2 μL of 10×attached BSA solution, 2 μL of 10 mM dNTPs (final concentration, 1 mM), 2 μL of 1 U/μL Phi29 Polymerase (final concentration, 0.1 U/μL), and 4 μL of water (20 μL in total).

### Preparation of b1 to b5 (Table 5)

A mixture was prepared with 2 μL of one of 100 nM DNA templates (1), (3), (4), and (5) (see Table 5) (final concentration, 10 nM), 2 μL of 400 nM DNA template (2) (final concentration, 40 nM), 2 μL of 120 nM DNA primer (1) (final concentration, 12 nM), 2 μL of 480 nM DNA primer (7) (final concentration, 48 nM), 2 μL of 10 x attached buffer, 2 μL of 10 x attached BSA solution, 2 μL of 10 mM dNTPs (final concentration, 1 mM), 2 μL of 1 U/μL Phi29 Polymerase (final concentration, 0.1 U/μL), 10 nM target DNA (1) (final concentration, 1 nM), and 2 μL of water (20 μL in total).

### Preparation of cl to c5 (Table 5)

A mixture was prepared with 2 μL of one of 100 nM DNA templates (1), (3), (4), and (5) (see Table 5) (final concentration, 10 nM), 2 μL of 400 nM DNA template (2) (final concentration, 40 nM), 2 μL of 120 nM DNA primer (1) (final concentration, 12 nM), 2 μL of 480 nM DNA primer (7) (final concentration, 48 nM), 2 μL of 10×attached buffer, 2 μL of 10 x attached BSA solution, 2 μL of 10 mM dNTPs (final concentration, 1 mM), 2 μL of 1 U/μL Phi29 Polymerase (final concentration, 0.1 U/μL), 2 μL of 10 nM double-stranded DNA (target DNA (1) and complementary DNA) (final concentration, 1 nM), and 2 μL of water (20 μL in total).

### Preparation of d1 to d5 (Table 5)

A mixture was prepared with 2 μL of one of 100 nM DNA templates (1), (3), (4), and (5) (see Table 5) (final concentration, 10 nM), 2 μL of 400 nM DNA template (2) (final concentration, 40 nM), 2 μL of 120 nM DNA primer (1) (final concentration, 12 nM), 2 μL of 480 nM DNA primer (7) (final concentration, 48 nM), 2 μL of 10×attached buffer, 2 μL of 10×attached BSA solution, 2 μL of 10 mM dNTPs (final concentration, 1 mM), 2 μL of 1 U/μL Phi29 Polymerase (final concentration, 0.1 U/μL), 10 nM complementary DNA (final concentration, 1 nM), and 2 μL of water (20 μL in total).

### [Example 4] Optimization of Primer for Ternary Complex

### Preparation of a1 to a5 (Table 7)

A mixture was prepared with 2 μL of one of 400 nM DNA templates (6) to (9) (see Table 7) (final concentration, 40 nM), 2 μL of one of 480 nM DNA primers (1) and (8) to (11) (see Table 7) (final concentration, 48 nM), 2 μL of 10×attached buffer, 2 μL of 10×attached BSA solution, 2 μL of 10 mM dNTPs (final concentration, 1 mM), 2 μL of 1 U/μL Phi29 Polymerase (final concentration, 0.1 U/μL), and 8 μL of water (20 μL in total).

### Preparation of b1 to b5 (Table 7)

A mixture was prepared with 2 μL of one of 400 nM DNA templates (6) to (9) (see Table 7) (final concentration, 40 nM), 2 μL of one of 480 nM DNA primers (1) and (8) to (10) (see Table 7) (final concentration, 48 nM), 2 μL of 10×attached buffer, 2 μL of 10×attached BSA solution, 2 μL of 10 mM dNTPs (final concentration, 1 mM), 2 μL of 1 U/μL Phi29 Polymerase (final concentration, 0.1 U/μL), 2 μL of 800 nM target RNA (1) (final concentration, 80 nM), and 6 μL of water (20 μL in total).

### Preparation of c1 to c5 (Table 7)

A mixture was prepared with 2 μL of one of 400 nM DNA templates (6) to (9) (see Table 7) (final concentration, 40 nM), 2 μL of one of 480 nM DNA primers (1) and (8) to (10) (see Table 7) (final concentration, 48 nM), 2 μL of 10 x attached buffer, 2 μL of 10 x attached BSA solution, 2 μL of 10 mM dNTPs (final concentration, 1 mM), 2 μL of 1 U/μL Phi29 Polymerase (final concentration, 0.1 U/μL), 2 μL of 800 nM no target RNA (final concentration, 80 nM), and 6 μL of water (20 μL in total).

[3] Reaction

The solutions prepared as described above were incubated at 37° C. for 2 hours. [4] Detection Visual Detection

To 8 μL of each reaction solution, 2 μL of 5×PBS153NM buffer (50 mM HP0₄²⁻, 730 mM Cl⁻, 765 mM Na⁺, 13.5 mM K⁺, 12.5 mM Mg²⁺; pH 7.4) was added.

With 10 μL of the mixed solution, 2 μL of a fluorescent dye (30 μM ThT derivative (THT-HE, JP 2016-079132 A) solution in 1×PBS153NM buffer; final concentration (5 μM)) was mixed, and then the resulting mixture was incubated at 25° C. for 30 minutes.

The prepared solution was subjected to irradiation using a 410-nm UV lamp. A photograph was taken with a camera equipped with a cut-off filter (which cuts off wavelengths shorter than 460 nm).

### Measurement Using Fluorescence Spectrophotometer

To 50 μL of the reaction solution of 4., 20 μL of 5×PBS153NM buffer and 30 μL of water were added.

With 100 μL of the mixed solution, 20 μL of a fluorescent dye (30 μM ThT derivative solution in 1×PBS153NM buffer; final concentration (5 μM)) was mixed, and then the resulting mixture was incubated at 25° C. for 30 minutes.

Using a fluorescence spectrophotometer, the fluorescence intensity of the solution was measured under the following conditions: excitation wavelength, 412 nm; measurement wavelength, 430 nm to 650 nm.

### Results

### [Example 1] One-base Mismatch of Primer

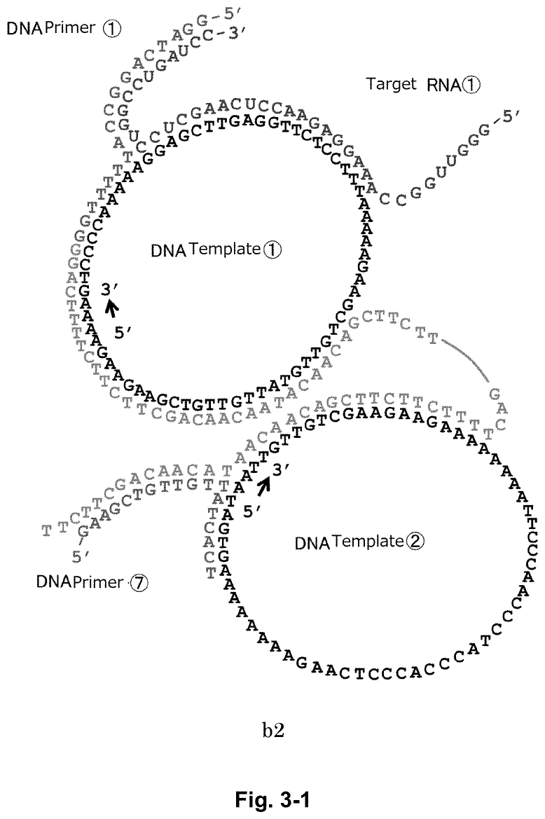

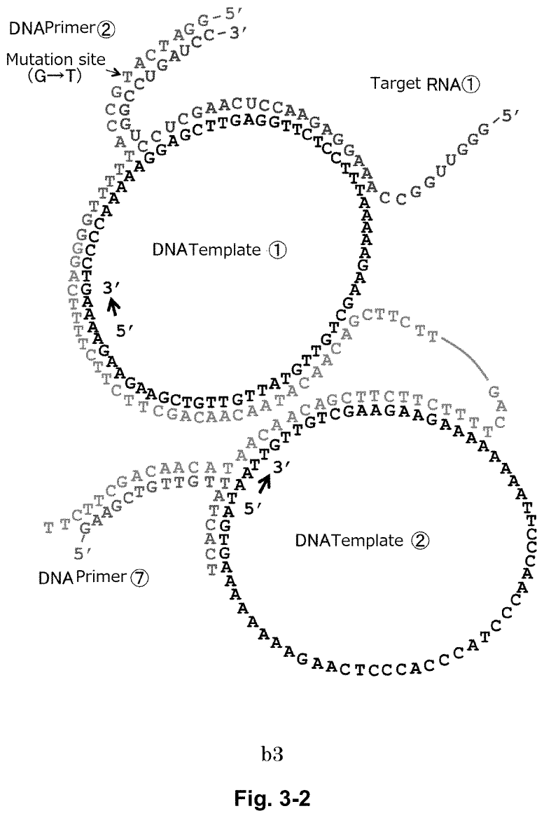

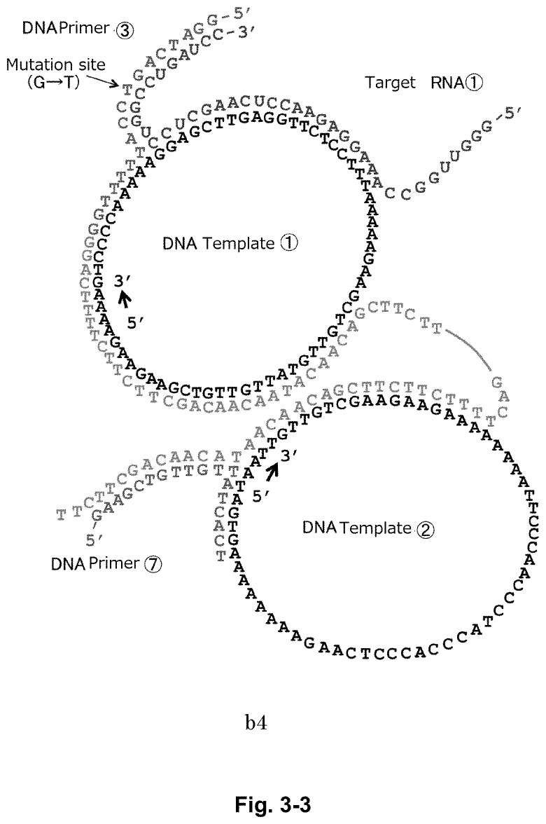

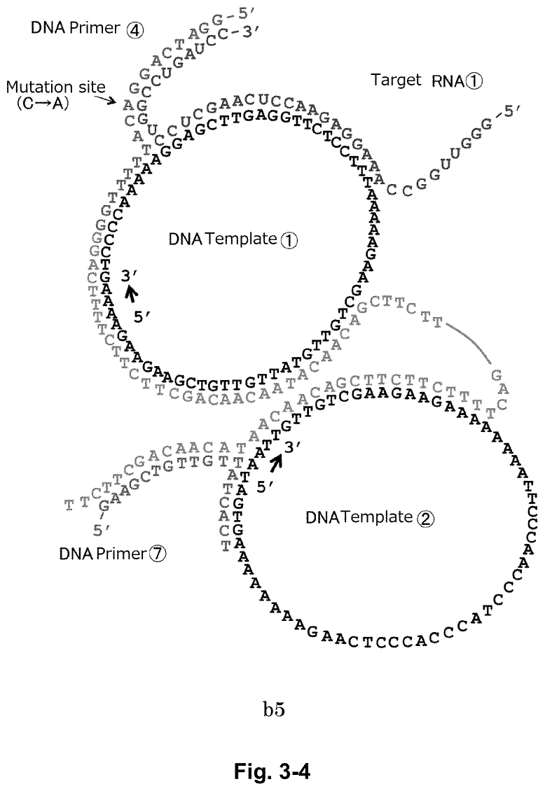

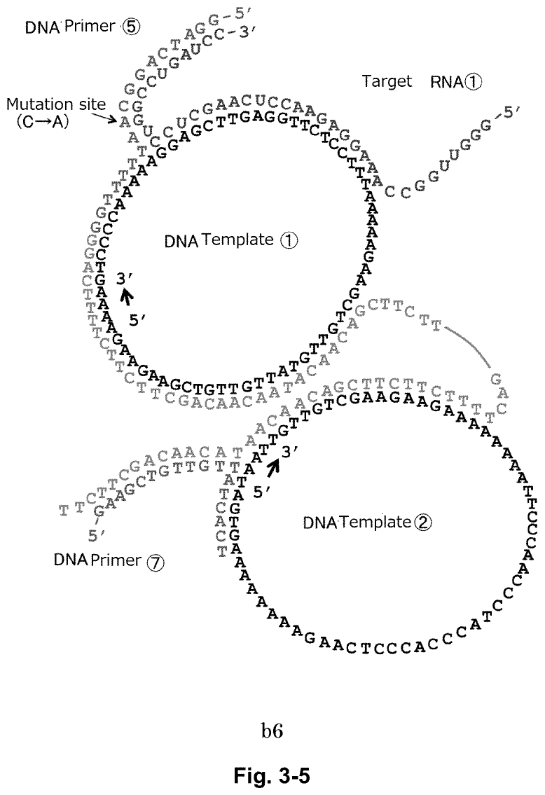

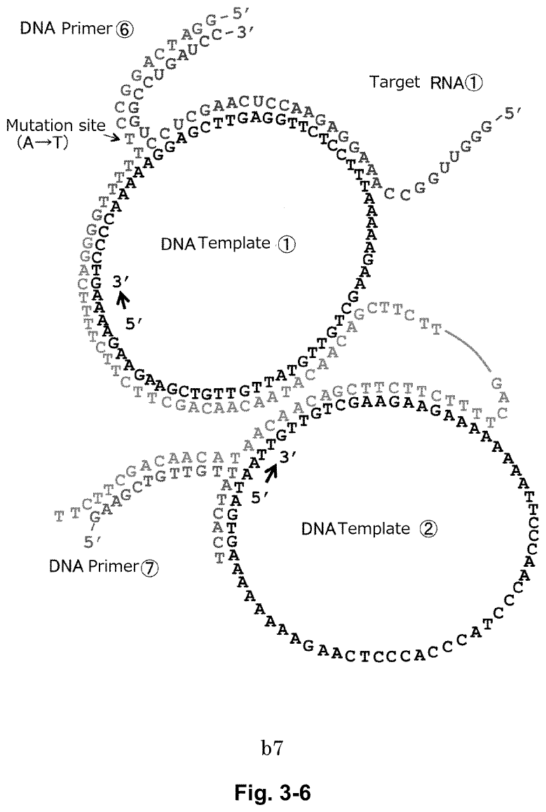

For each reaction composition in Table 2, the DNA primer (1) and the target RNA (1) were designed such that a one-base mismatch is present at a site of hybridization (FIG. 3), and changes in the progress of the reaction were studied.

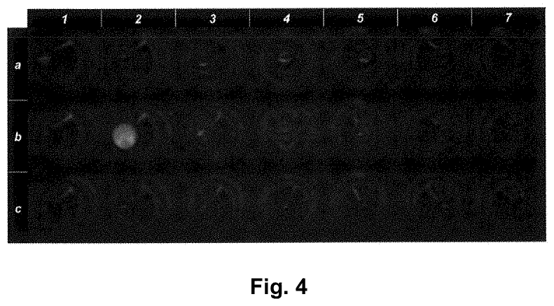

As a result, as shown in FIG. 4, the progress of the reaction was found to be more remarkably inhibited as the mismatch is located closer to the root of the ternary complex of the DNA primer (1), the target RNA (1), and the template DNA (1).

TABLE 2

| | a1 | a2 | a3 | a4 | a5 | a6 | a7 |
|---|---|---|---|---|---|---|---|
| Target RNA\textcircled{1} | − | − | − | − | − | − | − |
| No Target RNA | − | − | − | − | − | − | − |
| DNA Primer\textcircled{1} | − | + | − | − | − | − | − |
| DNA Primer\textcircled{2} | − | − | + | − | − | − | − |
| DNA Primer\textcircled{3} | − | − | − | + | − | − | − |
| DNA Primer\textcircled{4} | − | − | − | − | + | − | − |
| DNA Primer\textcircled{5} | − | − | − | − | − | + | − |
| DNA Primer\textcircled{6} | − | − | − | − | − | − | + |
| DNA Template\textcircled{1} | + | + | + | + | + | + | + |
| DNA Primer\textcircled{7} | + | + | + | + | + | + | + |
| DNA Template\textcircled{2} | + | + | + | + | + | + | + |
| | b1 | b2 | b3 | b4 | b5 | b6 | b7 |
| Target RNA\textcircled{1} | + | + | + | + | + | + | + |
| No Target RNA | − | − | − | − | − | − | − |
| DNA Primer\textcircled{1} | − | + | − | − | − | − | − |
| DNA Primer\textcircled{2} | − | − | + | − | − | − | − |
| DNA Primer\textcircled{3} | − | − | − | + | − | − | − |
| DNA Primer\textcircled{4} | − | − | − | − | + | − | − |
| DNA Primer\textcircled{5} | − | − | − | − | − | + | − |
| DNA Primer\textcircled{6} | − | − | − | − | − | − | + |
| DNA Template\textcircled{1} | + | + | + | + | + | + | + |
| DNA Primer\textcircled{7} | + | + | + | + | + | + | + |
| DNA Template\textcircled{2} | + | + | + | + | + | + | + |
| | c1 | c2 | c3 | c4 | c5 | c6 | c7 |
| Target RNA\textcircled{1} | − | − | − | − | − | − | − |
| No Target RNA | + | + | + | + | + | + | + |
| DNA Primer\textcircled{1} | − | + | − | − | − | − | − |
| DNA Primer\textcircled{2} | − | − | + | − | − | − | − |
| DNA Primer\textcircled{3} | − | − | − | + | − | − | − |
| DNA Primer\textcircled{4} | − | − | − | − | + | − | − |
| DNA Primer\textcircled{5} | − | − | − | − | − | + | − |
| DNA Primer\textcircled{6} | − | − | − | − | − | − | + |
| DNA Template\textcircled{1} | + | + | + | + | + | + | + |
| DNA Primer\textcircled{7} | + | + | + | + | + | + | + |
| DNA Template\textcircled{2} | + | + | + | + | + | + | + |

### [Example 2] Difference between DNA/RNA Targets, and One-base Mismatch

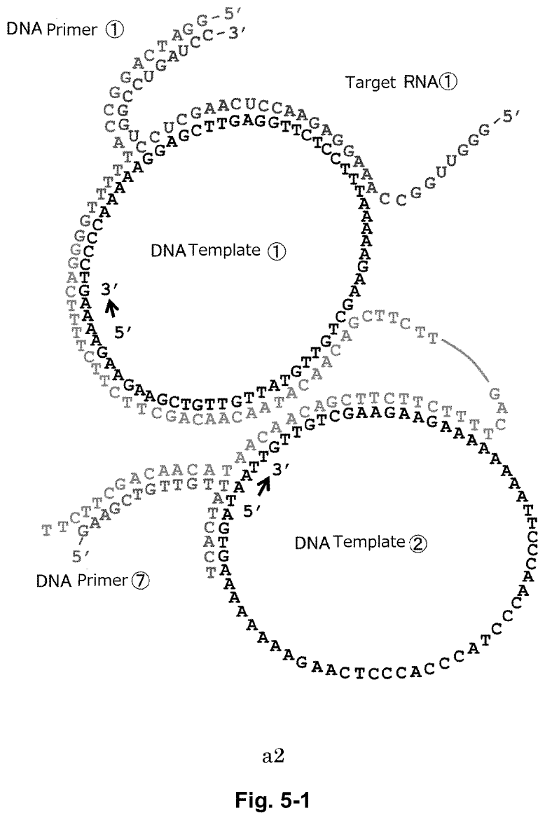

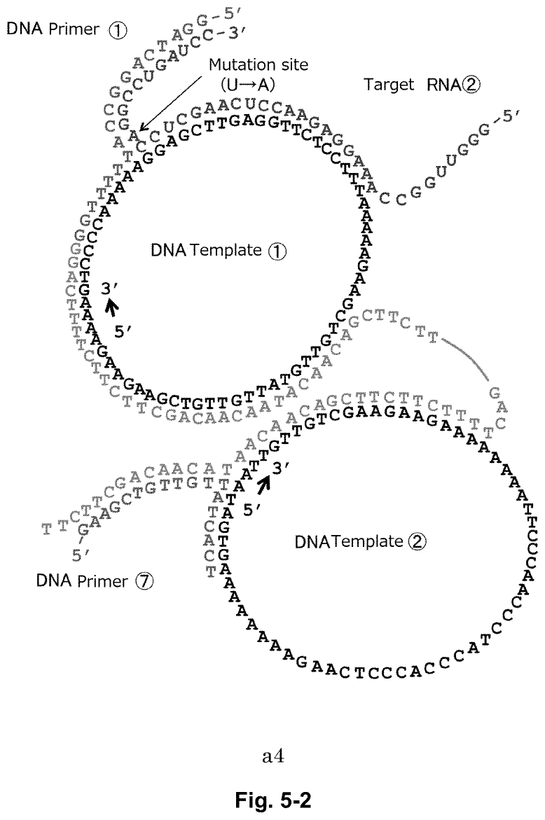

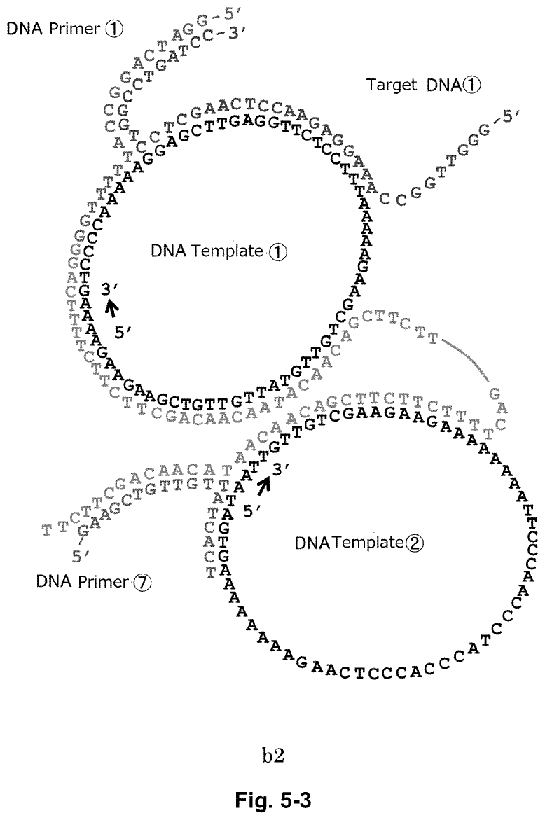

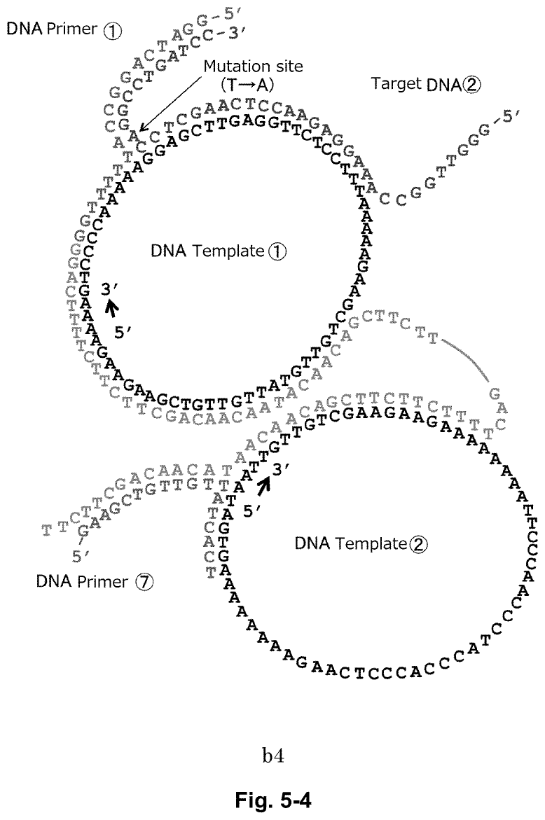

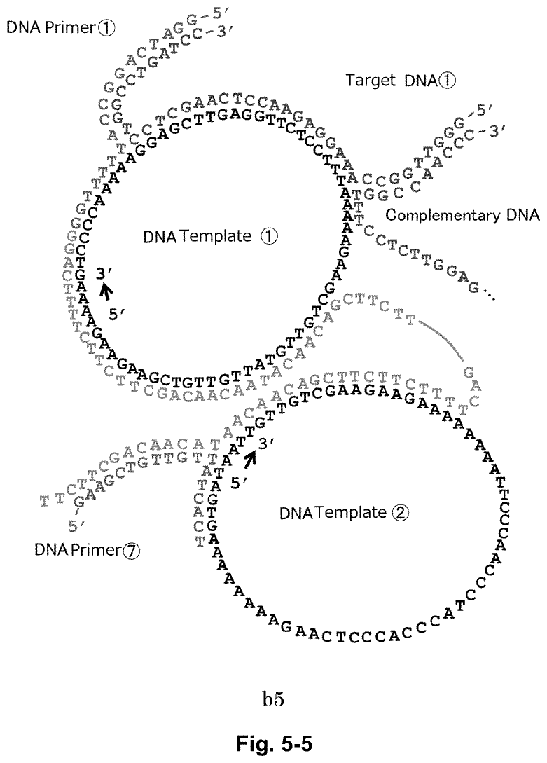

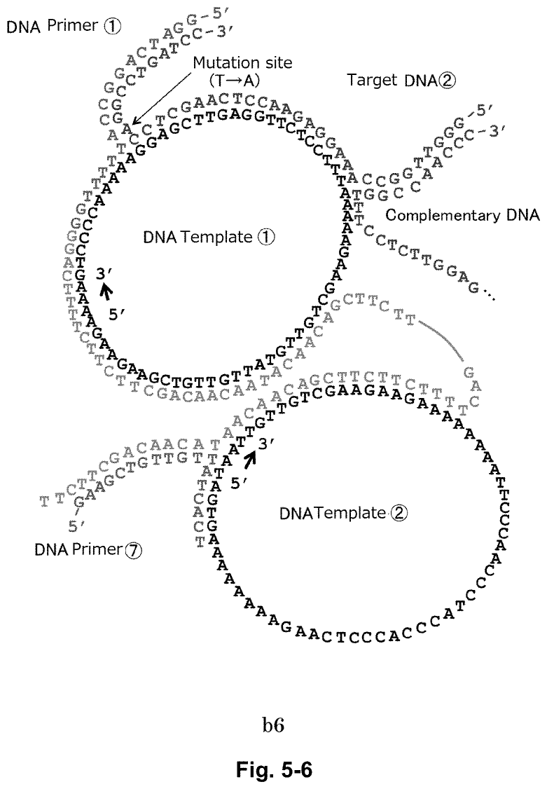

For each reaction composition in Table 3, a target prepared by mutating a site at the root portion of the ternary complex to introduce a mismatch (30→A) to the target RNA (1) was used to study the progress of the reaction (FIG. 5). In addition, whether the present method allows the detection also in a case where the target is DNA was studied.

As a result, it was found that, when there is the mismatch in the target RNA (FIG. 6 a4), the progress of the reaction was remarkably inhibited.

Further, it was found that the detection is possible also when the target is DNA, as shown by b3 in FIG. 6. It was found that, as in the case of the target RNA, the target DNA having the one-base mismatch (FIG. 6 b4) similarly shows remarkable inhibition of the reaction. In general, DNA tends to be present as a double strand in the body. In view of this, a study was carried out to see whether the detection is possible even when the target is in the form of a double strand of a target DNA and complementary DNA having a sequence complementary thereto (FIG. 6 b5). As a result, although the fluorescence intensity was weaker than that in the case of the single-stranded DNA, an evident luminescence was found relative to the non-target DNA (FIG. 6 b3) or the target DNA (2) having a one-base mismatch (FIG. 6 b4). Thus, it was suggested that the detection is sufficiently possible. On the other hand, it was found that the progress of the reaction is inhibited in the case where the target is in the form of a double strand of the target DNA (2) having a one-base mismatch and complementary DNA (FIG. 6 b6.)

TABLE 3

| | a1 | a2 | a3 | a4 | a5 | a6 |
|---|---|---|---|---|---|---|
| Target RNA\textcircled{1} | − | + | − | − | | |
| No Target RNA | − | − | + | − | | |
| Target RNA\textcircled{2} | − | − | − | + | | |
| Target DNA\textcircled{1} | − | − | − | − | | |
| Target DNA\textcircled{2} | − | − | − | − | | |
| Complementary DNA | − | − | − | − | | |
| DNA Primer\textcircled{1} | + | + | + | + | | |
| DNA Template\textcircled{1} | + | + | + | + | | |
| DNA Primer\textcircled{7} | + | + | + | + | | |
| DNA Template\textcircled{2} | + | + | + | + | | |
| | b1 | b2 | b3 | b4 | b5 | b6 |
| Target RNA\textcircled{1} | − | − | − | − | − | − |
| No Tarqet RNA | − | − | − | − | − | − |
| Target RNA\textcircled{2} | − | − | − | − | − | − |
| Target DNA\textcircled{1} | − | + | − | − | + | − |
| Target DNA\textcircled{2} | − | − | + | − | + | + |
| Complementary DNA | − | − | − | + | − | + |
| DNA Primer\textcircled{1} | + | + | + | + | + | + |
| DNA Template\textcircled{1} | + | + | + | + | + | + |
| DNA Primer\textcircled{7} | + | + | + | + | + | + |
| DNA Template\textcircled{2} | + | + | + | + | + | + |

With the reaction compositions in Table 4, a comparison of the fluorescence was made using a fluorescence spectrophotometer between the cases where a double-stranded DNA was targeted and the cases where a single-stranded DNA was targeted, to study whether fluorescence having the same characteristics is obtained or not (FIG. 7).

As a result, each fluorescence spectrum had almost the same peak top wavelength, and the fluorescence intensity increased as the reaction proceeded. It was thus suggested that the fluorescence has the same characteristics.

TABLE 4

| | b1 | a2 | b2 | b4 | b5 | b6 |
|---|---|---|---|---|---|---|
| Target RNA\textcircled{1} | − | + | − | − | − | − |
| Target DNA\textcircled{1} | − | − | + | − | + | − |
| Target DNA\textcircled{2} | − | − | − | + | − | + |
| Complementary DNA | − | − | − | − | + | + |
| DNA Primer\textcircled{1} | + | + | + | + | + | + |
| DNA Template\textcircled{2} | + | + | + | + | + | + |
| DNA Primer\textcircled{7} | + | + | + | + | + | + |
| DNA Template\textcircled{2} | + | + | + | + | + | + |

### [Example 3] Optimization of Circular Template for Double-stranded DNA Target

In cases where a double-stranded DNA is to be targeted, the double strand needs to be dissociated for formation of the ternary complex. In order to promote binding between the target DNA and the DNA template, a sequence that hybridizes with the complementary sequence (Table 5: complementary DNA) of the target DNA was extended by three-base increments in the DNA template (1) (FIG. 8).

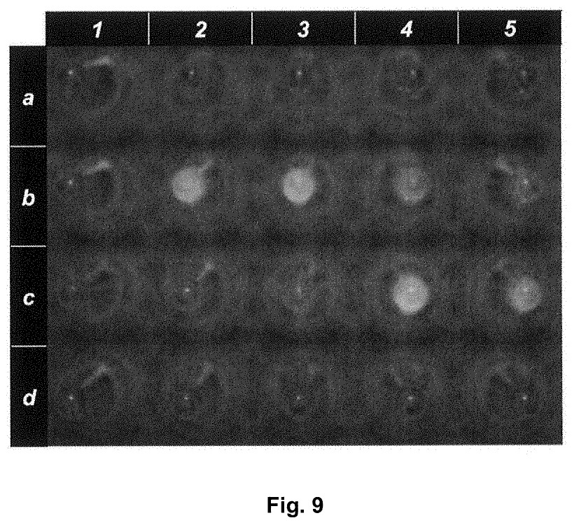

As a result of performing the reaction with each reaction composition in Table 5 using this, in the cases of ssDNA (target DNA (1)), it was found, as shown in FIG. 9, that inhibition of the progress of the reaction occurred as the insertion number increased. This is thought to be due to the fact that, when complementary DNA is absent, stems are easily formed in the DNA template, leading to inhibition of the binding between the template and the target.

On the other hand, in the cases of dsDNA, it was found that the progress of the reaction was promoted as the insertion number increased. Thus, in cases where the target is dsDNA, the reaction can be promoted by including, in the template, an appropriate number of sequences complementary to complementary DNA.

TABLE 5

| | a1 | a2 | a3 | a4 | a5 |
|---|---|---|---|---|---|
| Target DNA\textcircled{1} | − | − | − | − | − |
| Complementary DNA | − | − | − | − | − |
| DNA Primer\textcircled{1} | + | + | + | + | + |
| DNA Template\textcircled{1} | − | + | − | − | − |
| DNA Template\textcircled{3} | − | − | + | − | − |
| DNA Template\textcircled{4} | − | − | − | + | − |
| DNA Template\textcircled{5} | − | − | − | − | + |
| DNA Primer\textcircled{7} | + | + | + | + | + |
| DNA Template\textcircled{2} | + | + | + | + | + |
| | b1 | b2 | b3 | b4 | b5 |
| Target DNA\textcircled{1} | + | + | + | + | + |
| Complementary DNA | − | − | − | − | − |
| DNA Primer\textcircled{1} | + | + | + | + | + |
| DNA Template\textcircled{1} | − | + | − | − | − |
| DNA Template\textcircled{3} | − | − | + | − | − |
| DNA Template\textcircled{4} | − | − | − | + | − |
| DNA Template\textcircled{5} | − | − | − | − | + |
| DNA Primer\textcircled{7} | + | + | + | + | + |
| DNA Template\textcircled{2} | + | + | + | + | + |
| | c1 | c2 | c3 | c4 | c5 |
| Target DNA\textcircled{1} | + | + | + | + | + |
| Complementary DNA | + | + | + | + | + |
| DNA Primer\textcircled{1} | + | + | + | + | + |
| DNA Template\textcircled{1} | − | + | − | − | − |
| DNA Template\textcircled{3} | − | − | + | − | − |
| DNA Template\textcircled{4} | − | − | − | + | − |
| DNA Template\textcircled{5} | − | − | − | − | + |
| DNA Primer\textcircled{7} | + | + | + | + | + |
| DNA Template\textcircled{2} | + | + | + | + | + |
| | d1 | d2 | d3 | d4 | d5 |
| Target DNA\textcircled{1} | − | − | − | − | − |
| Complementary DNA | + | + | + | + | + |
| DNA Primer\textcircled{1} | + | + | + | + | + |
| DNA Template\textcircled{1} | − | + | − | − | − |
| DNA Template\textcircled{3} | − | − | + | − | − |
| DNA Template\textcircled{4} | − | − | − | + | − |
| DNA Template\textcircled{5} | − | − | − | − | + |
| DNA Primer\textcircled{7} | + | + | + | + | + |
| DNA Template\textcircled{2} | + | + | + | + | + |

### [Example 4] Optimization of Primer for Ternary Complex

It has been known that the reaction does not proceed when the DNA primer is 17mer (DNA primer (8)). Stability of a double-stranded DNA depends on the length of the DNA and its sequence. In view of this, optimization of the DNA primer was carried out by changing the sequence focusing on the intermediate stability between the stabilities of the DNA primer (1) (18mer), which allows the reaction to proceed, and the DNA primer (8), which does not allow the reaction to proceed.

The optimization in this experiment was carried out by using the DNA primer (1) as a base, and changing the two residues in its 3′-end. According to the nearest-neighbor thermodynamics, the stabilities of the double strands are in the following order: DNA primers (1) >(11)>(10)>(9)>(8) (Table 6).

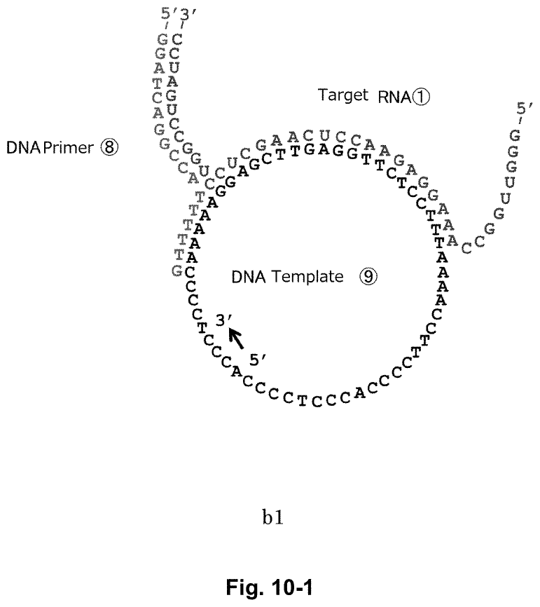

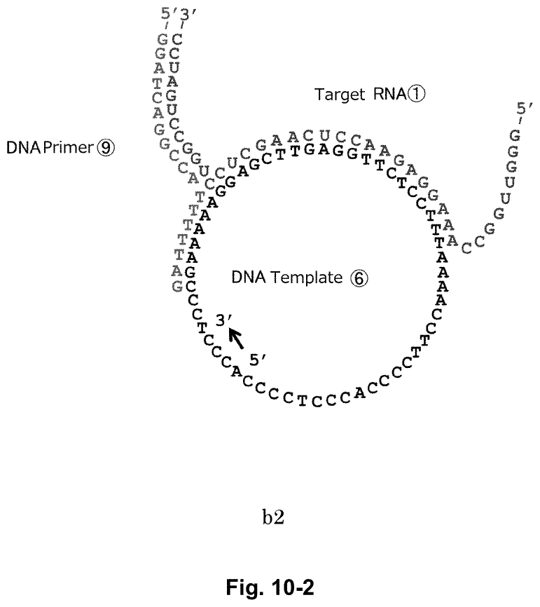

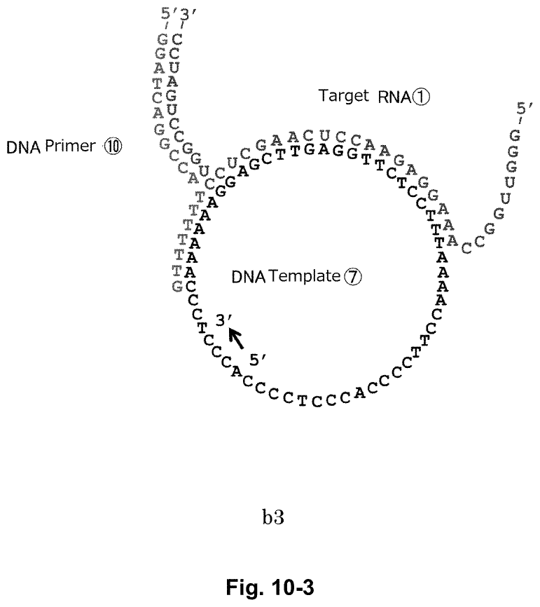

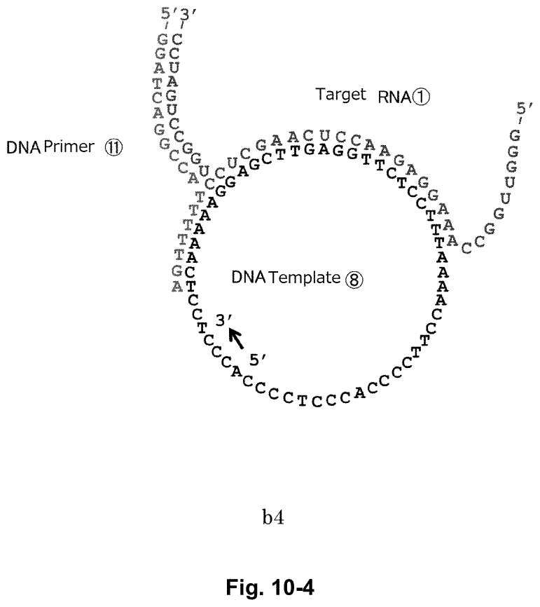

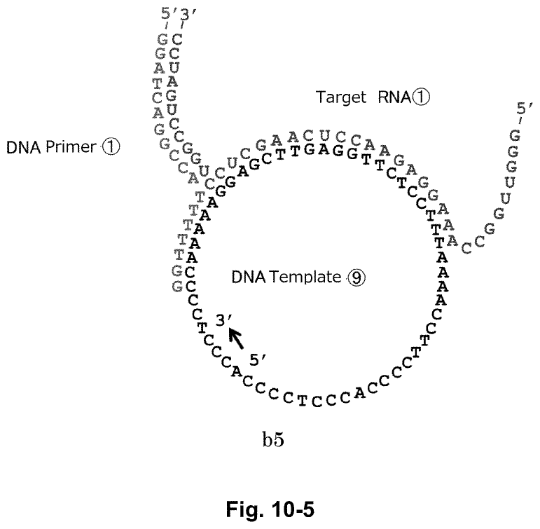

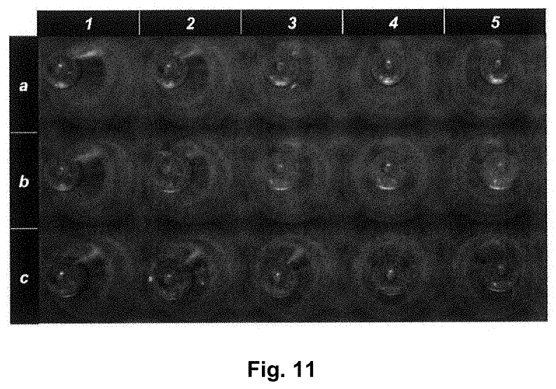

As a result of performing the reaction with each reaction composition in Table 7 (FIG. 10), as shown in FIG. 11, the DNA primer (9) did not allow the reaction to proceed, but the DNA primers (10) and (11) allowed the reaction to proceed. Thus, the border that determines whether the reaction proceeds or not was found to exist between the stabilities of the DNA primers (9) and (10). Accordingly, it was thought that ΔG⁰₃₇ of the primer-template double strand is preferably less than −3.8.

TABLE 6
Δ G^{o}₃₇ of primer-tenplate double strand
calculated from Nearestneighbor thermodynamics

| | Δ G^{o}₃₇ |
|---|---|
| Primer/Template | (kcal/mol) |
| DNA Primer (8)/DNA Template (9) | −3.1 |
| DNA Primer (9)/DNA Template (6) | −3.8 |
| DNA Primer (10)/DNA Template (7) | −4.3 |
| DNA Primer (11)/DNA Template (8) | −4.6 |
| DNA Primer (1)/DNA Template (9) | −5.2 |

TABLE 7

| | a1 | a2 | a3 | a4 | a5 |
|---|---|---|---|---|---|
| Target RNA\textcircled{1} | − | − | − | − | − |
| No Target RNA | − | − | − | − | − |
| DNA Primer\textcircled{8} | + | − | − | − | − |
| DNA Template\textcircled{9} | + | − | − | − | + |
| DNA Primer\textcircled{9} | − | + | − | − | − |
| DNA Template\textcircled{6} | − | + | − | − | − |
| DNA Primer\textcircled{10} | − | − | + | − | − |
| DNA Template\textcircled{7} | − | − | + | − | − |
| DNA Primer\textcircled{11} | − | − | − | + | − |
| DNA Template\textcircled{8} | − | − | − | + | − |
| DNA Primer\textcircled{1} | − | − | − | − | + |
| | b1 | b2 | b3 | b4 | b5 |
| Target RNA\textcircled{1} | + | + | + | + | + |
| No Target RNA | − | − | − | − | − |
| DNA Primer\textcircled{8} | + | − | − | − | − |
| DNA Template\textcircled{9} | + | − | − | − | + |
| DNA Primer\textcircled{9} | − | + | − | − | − |
| DNA Template\textcircled{6} | − | + | − | − | − |
| DNA Primer\textcircled{10} | − | − | + | − | − |
| DNA Template\textcircled{7} | − | − | + | − | − |
| DNA Primer\textcircled{11} | − | − | − | + | − |
| DNA Template\textcircled{8} | − | − | − | + | − |
| DNA Primer\textcircled{1} | − | − | − | − | + |
| | c1 | c2 | c3 | c4 | c5 |
| Target RNA\textcircled{1} | − | − | − | − | − |
| No Target RNA | + | + | + | + | + |
| DNA Primer\textcircled{8} | + | − | − | − | − |
| DNA Template\textcircled{9} | + | − | − | − | + |
| DNA Primer\textcircled{9} | − | + | − | − | − |
| DNA Template\textcircled{6} | − | + | − | − | − |
| DNA Primer\textcircled{10} | − | − | + | − | − |
| DNA Template\textcircled{7} | − | − | + | − | − |
| DNA Primer\textcircled{11} | − | − | − | + | − |
| DNA Template\textcircled{8} | − | − | − | + | − |
| DNA Primer\textcircled{1} | − | − | − | − | + |

### [Example 5] Detection of Single Nucleotide Polymorphism in Long-chain DNA

For detection of single nucleotide polymorphisms (SNPs) in a long-chain DNA, the following experiment was carried out. FIG. 12 shows the reaction scheme. As the target sequence containing a SNP, a sequence of 1 kbp (500 bp each of the sequences before and after the mutation site) containing a portion of the CYP2C19 gene, which encodes cytochrome P450, which portion contains a SNP at position 636 or a SNP at position 681, was obtained by PCR amplification from a genomic sample of human cultured cells (HepG2).

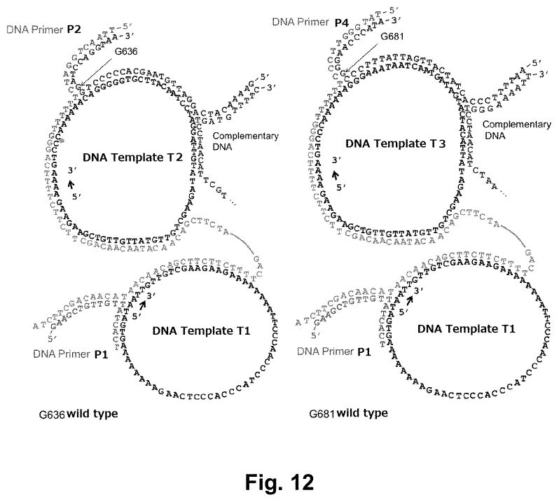

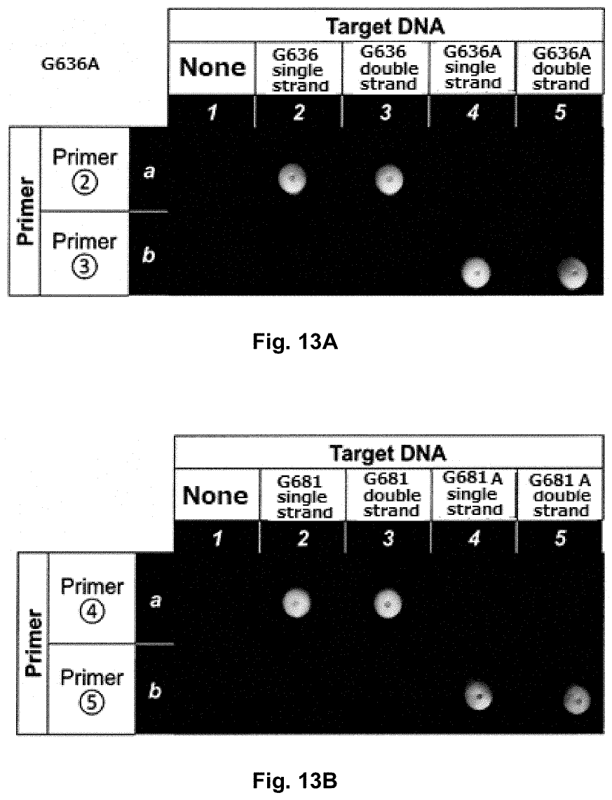

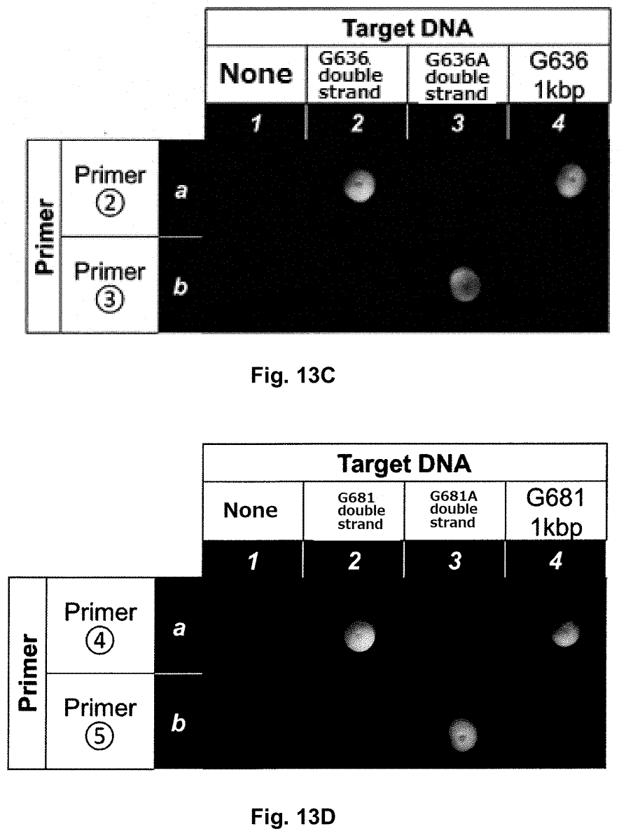

The following reagents were prepared, and extension reaction was carried out by adding the above target sequence thereto and performing incubation at 37° C. for 2 hours in order to detect a mutation (mutation from G to A) of the base at position 636 (G636) in the CYP2C19 gene, which encodes cytochrome P450 (FIG. 13(A) and (C)), or a mutation (mutation from G to A) of the base at position 681 (G681) in the CYP2C19 gene (FIG. 13(B) and (D)). To 8 μL of each reaction solution, 2 μL of 5×PBS153NM buffer (50 mM HP0₄²⁻, 730 mM Cr, 765 mM Nat, 13.5 mM K⁺, 12.5 mM Mg²+; pH 7.4) was added. With 10 μL of the mixed solution, 2 μL of a fluorescent dye (30 μM ThT derivative (THT-HE, JP 2016-079132 A) solution in 1 x PBS153NM buffer; final concentration (5 μM)) was mixed, and then the resulting mixture was incubated at 25° C. for 30 minutes. The prepared solution was subjected to irradiation using a 410-nm UV lamp. A photograph was taken with a camera equipped with a cut-off filter (which cuts off wavelengths shorter than 460 nm).

### Preparation of FIG. 13(A) a1 to a5

A mixture was prepared with 2 μL of 100 nM DNA template T2 (final concentration, 10 nM), 2 μL of 400 nM DNA template T1 (final concentration, 40 nM), 2 μL of 120 nM primer P2 (final concentration, 12 nM), 2 μL of 480 nM DNA primer P1 (final concentration, 48 nM), 2 μL of 10×attached buffer, 2 μL of 10×attached BSA solution, 2 μL of 10 mM dNTPs (final concentration, 1 mM), 2 μL of 1 U/μL Phi29 Polymerase (final concentration, 0.1 U/μL), 2 μL of water or 10 nM target DNA (40mer; annealing of double-stranded DNA was carried out in advance; final concentration, 1 nM), and 2 μL of water (20 μL in total).

Preparation of FIG. 13(A) b1 to b5

A mixture was prepared with 2 μL of 100 nM DNA template T2 (final concentration, 10 nM), 2 μL of 400 nM DNA template T1 (final concentration, 40 nM), 2 μL of 120 nM primer P3 (final concentration, 12 nM), 2 μL of 480 nM DNA primer P1 (final concentration, 48 nM), 2 μL of 10×attached buffer, 2 μL of 10 x attached BSA solution, 2 μL of 10 mM dNTPs (final concentration, 1 mM), 2 μL of 1 U/μL Phi29 Polymerase (final concentration, 0.1 U/μL), 2 μL of water or 10 nM target DNA (40mer; annealing of double-stranded DNA was carried out in advance; final concentration, 1 nM), and 2 μL of water (20 μL in total).

### Preparation of FIG. 13(B) a1 to a5

A mixture was prepared with 2 μL of 100 nM DNA template T3 (final concentration, 10 nM), 2 μL of 400 nM DNA template T1 (final concentration, 40 nM), 2 μL of 120 nM primer P4 (final concentration, 12 nM), 2 μL of 480 nM DNA primer P1 (final concentration, 48 nM), 2 μL of 10×attached buffer, 2 μL of 10×attached BSA solution, 2 μL of 10 mM dNTPs (final concentration, 1 mM), 2 μL of 1 U/μL Phi29 Polymerase (final concentration, 0.1 U/μL), 2 μL of water or 10 nM target DNA (40mer; annealing of double-stranded DNA was carried out in advance; final concentration, 1 nM), and 2 μL of water (20 μL in total).

### Preparation of FIGS. 13(B) b1 to b5

A mixture was prepared with 2 μL of 100 nM DNA template T3 (final concentration, 10 nM), 2 μL of 400 nM DNA template T1 (final concentration, 40 nM), 2 μL of 120 nM primer P5 (final concentration, 12 nM), 2 μL of 480 nM DNA primer P1 (final concentration, 48 nM), 2 μL of 10×attached buffer, 2 μL of 10×attached BSA solution, 2 μL of 10 mM dNTPs (final concentration, 1 mM), 2 μL of 1 U/μL Phi29 Polymerase (final concentration, 0.1 U/μL), 2 μL of water or 10 nM target DNA (40mer; annealing of double-stranded DNA was carried out in advance; final concentration, 1 nM), and 2 μL of water (20 μL in total).

### Preparation of FIG. 13(C) a1 to a5

A mixture was prepared with 2 μL of 100 nM DNA template T2 (final concentration, 10 nM), 2 μL of 400 nM DNA template T1 (final concentration, 40 nM), 2 μL of 120 nM primer P2 (final concentration, 12 nM), 2 μL of 480 nM DNA primer P1 (final concentration, 48 nM), 2 μL of 10 x attached buffer, 2 μL of 10 x attached BSA solution, 2 μL of 10 mM dNTPs (final concentration, 1 mM), 2 μL of 1 U/μL Phi29 Polymerase (final concentration, 0.1 U/μL), 2 μL of water or 10 nM target DNA (40mer, or annealing of double-stranded DNA amplified from the HepG2 gene was carried out in advance; final concentration, 1 nM), and 2 μL of water (20 μL in total).

### Preparation of FIG. 13(C) b1 to b5

A mixture was prepared with 2 μL of 100 nM DNA template T2 (final concentration, 10 nM), 2 μL of 400 nM DNA template T1 (final concentration, 40 nM), 2 μL of 120 nM primer P3 (final concentration, 12 nM), 2 μL of 480 nM DNA primer P1 (final concentration, 48 nM), 2 μL of 10×attached buffer, 2 μL of 10 x attached BSA solution, 2 μL of 10 mM dNTPs (final concentration, 1 mM), 2 μL of 1 U/μL Phi29 Polymerase (final concentration, 0.1 U/μL), 2 μL of water or 10 nM target DNA (40mer, or annealing of double-stranded DNA amplified from the HepG2 gene was carried out in advance; final concentration, 1 nM), and 2 μL of water (20 μL in total).

### Preparation of FIG. 13(D) a1 to a5

A mixture was prepared with 2 μL of 100 nM DNA template T3 (final concentration, 10 nM), 2 μL of 400 nM DNA template T1 (final concentration, 40 nM), 2 μL of 120 nM primer P4 (final concentration, 12 nM), 2 μL of 480 nM DNA primer P1 (final concentration, 48 nM), 2 μL of 10×attached buffer, 2 μL of 10 x attached BSA solution, 2 μL of 10 mM dNTPs (final concentration, 1 mM), 2 μL of 1 U/μL Phi29 Polymerase (final concentration, 0.1 U/μL), 2 μL of water or 10 nM target DNA (40mer, or annealing of double-stranded DNA amplified from the HepG2 gene was carried out in advance; final concentration, 1 nM), and 2 μL of water (20 μL in total).

### Preparation of FIG. 13(D) b1to b5

A mixture was prepared with 2 μL of 100 nM DNA template T3 (final concentration, 10 nM), 2 μL of 400 nM DNA template T1 (final concentration, 40 nM), 2 μL of 120 nM primer P5 (final concentration, 12 nM), 2 μL of 480 nM DNA primer P1 (final concentration, 48 nM), 2 μL of 10×attached buffer, 2 μL of 10×attached BSA solution, 2 μL of 10 mM dNTPs (final concentration, 1 mM), 2 μL of 1 U/μL Phi29 Polymerase (final concentration, 0.1 U/μL), 2 μL of water or 10 nM target DNA (40mer, or annealing of double-stranded DNA amplified from the HepG2 gene was carried out in advance; final concentration, 1 nM), and 2 μL of water (20 μL in total).

TABLE 8
Sequences of Oligonucleotides

| | Length | | |
|---|---|---|---|
| | (mer) | Sequence (5′→3′) | Modification |
| DNA Primer P1 | 18 | GAAGCTGTTGTTATCACT (SEQ ID NO: 33) | |
| DNA Primer P2 | 18 | TTACGTGGATCTTTTTTG | |
| (Wild type) | | (SEQ ID NO: 34) | |
| DNA Primer P3 | 18 | TTACCTGGATTTTTTTTG | |
| (Mutant type) | | (SEQ ID NO: 35) | |
| DNA Primer P4 | 18 | TATGGGTTCCCTTTTTTG | |
| (Wild type) | | (SEQ ID NO: 36) | |
| DNA Primer P5 | 18 | TATGGGTTCCTTTTTTTG | |
| (Mutant type) | | (SEQ ID NO: 37) | |
| DNA Template T1 | 62 | CCCAACCCTACCCACCCTCAAGAAAAAAAAGTGATAATTGTTG | Circulated |
| (Second single | | TCGAAGAAGAAAAAAAATT | |
| strand circular DNA) | | (SEQ ID NO: 38) | |
| DNA Template T2 | 73 | CAAAAAACAGGGGGTGCTTACAATCCTAGGATTGTATAGAAGC | Circulated |
| (First single strand | | TGTTGTATTGTTGTCGAAGAAGAAAAGTCC | |
| circular DNA: G636) | | (SEQ ID NO: 39) | |
| DNA Template T3 | 73 | CAAAAAAGGGAAATAATCAATGATAGTACTATCATTTAGAAGC | Circulated |
| (First single strand | | TGTTGTATTGTTGTCGAAGAAGAAAAGTCC | |
| circular DNA: G681) | | (SEQ ID NO: 40) | |

[3] Detection of Single Nucleotide Polymorphism in Long-chain DNA

In the study of the optimized conditions, a 40mer single-stranded or double-stranded DNA was targeted. Regarding the base at position 636 in the CYP2C19 gene, for the single strand and the double-strand, the wild type (FIG. 13(A) a2, a3) could be distinguished from the mutant (FIG. 13(A) b4, b5). Further, regarding the base at position 681 in the CYP2C19 gene, the wild type (FIG. 13(B) a2, a3) could be distinguished from the mutant (FIG. 13(B) b4, b5).

The genomic sample of the human cultured cells (HepG2) was prepared by PCR amplification of a 1-kbp region (500 bp each of the sequences before and after the mutation site). As a result, the wild type could be confirmed for both G636 (FIG. 13(C) a4) and G681 (FIG. 13(D) a4). Thus, mutations can be confirmed even in cases where the target is a long-chain double-stranded DNA of 1 kbp.

### [Example 6] Detection of Mutant miRNAs Having Different Chain Lengths

For detection of 2-base addition to the 3′-end of miRNA, the following experiment was carried out. FIG. 16 shows the reaction scheme. miR-21CA, in Table 9, was prepared by addition of the two bases CA to the 3′-end of miR-21.

For detection of this mutation, the following reagents were prepared, and extension reaction was carried out by incubation at 37° C. for 2 hours. To 8 μL of each reaction solution, 2 μL of 5×PBS153NM buffer (50 mM HP0₄²⁻, 730 mM Cl⁻, 765 mM Nat, 13.5 mM K⁺, 12.5 mM Mg²⁺; pH 7.4) was added. With 10 μL of the mixed solution, 2 μL of a fluorescent dye (30 μM ThT derivative (THT-HE, JP 2016-079132 A) solution in 1×PBS153NM buffer; final concentration (5 μM)) was mixed, and then the resulting mixture was incubated at 25° C. for 30 minutes. The prepared solution was subjected to irradiation using a 410-nm UV lamp. A photograph was taken with a camera equipped with a cut-off filter (which cuts off wavelengths shorter than 460 nm).

### Preparation of FIGS. 17 a1 and a2

A mixture was prepared with 2 μL of 100 nM DNA template T4 (final concentration, 10 nM), 2 μL of 400 nM DNA template T1 (final concentration, 40 nM), 2 μL of water or 120 nM capture probe (1) (final concentration, 12 nM), 2 μL of 480 nM DNA primer P6 (final concentration, 48 nM), 2 μL of 10 x attached buffer, 2 μL of 10 x attached BSA solution, 2 μL of 10 mM dNTPs (final concentration, 1 mM), 2 μL of 1 U/μL Phi29 Polymerase (final concentration, 0.1 U/μL), and 4 μL of water (20 μL in total).

### Preparation of FIGS. 17 b1 and b2

A mixture was prepared with 2 μL of 100 nM DNA template T4 (final concentration, 10 nM), 2 μL of 400 nM DNA template T1 (final concentration, 40 nM), 2 μL of water or 120 nM capture probe (1) (final concentration, 12 nM), 2 μL of 480 nM DNA primer P6 (final concentration, 48 nM), 2 μL of 10×attached buffer, 2 μL of 10×attached BSA solution, 2 μL of 10 mM dNTPs (final concentration, 1 mM), 2 μL of 1 U/μL Phi29 Polymerase (final concentration, 0.1 U/μL), 2 μL of 10 nM miR-21 (final concentration, 1 nM), and 2 μL of water (20 μL in total).

### Preparation of FIG. 17 c1 and c2

A mixture was prepared with 2 μL of 100 nM DNA template T4 (final concentration, 10 nM), 2 μL of 400 nM DNA template T1 (final concentration, 40 nM), 2 μL of water or 120 nM capture probe (1) (final concentration, 12 nM), 2 μL of 480 nM DNA primer P6 (final concentration, 48 nM), 2 μL of 10 x attached buffer, 2 μL of 10×attached BSA solution, 2 μL of 10 mM dNTPs (final concentration, 1 mM), 2 μL of 1 U/μL Phi29 Polymerase (final concentration, 0.1 U/μL), 2 μL of 10 nM miR-21CA (final concentration, 1 nM), and 2 μL of water (20 μL in total).

### Preparation of FIG. 17 d1 and d2

A mixture was prepared with 2 μL of 100 nM DNA template T4 (final concentration, 10 nM), 2 μL of 400 nM DNA template T1 (final concentration, 40 nM), 2 μL of water or 120 nM capture probe (1) (final concentration, 12 nM), 2 μL of 480 nM DNA primer P6 (final concentration, 48 nM), 2 μL of 10×attached buffer, 2 μL of 10×attached BSA solution, 2 μL of 10 mM dNTPs (final concentration, 1 mM), 2 μL of 1 U/μL Phi29 Polymerase (final concentration, 0.1 U/μL), 2 μL of 10 nM miR-221 (final concentration, 1 nM), and 2 μL of water (20 μL in total).

### [Example 7] Detection of miRNA Having Single-base Substitution Mutation

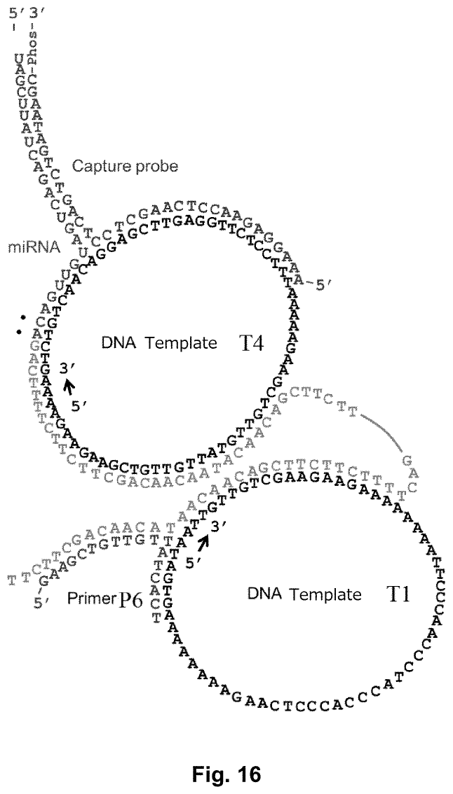

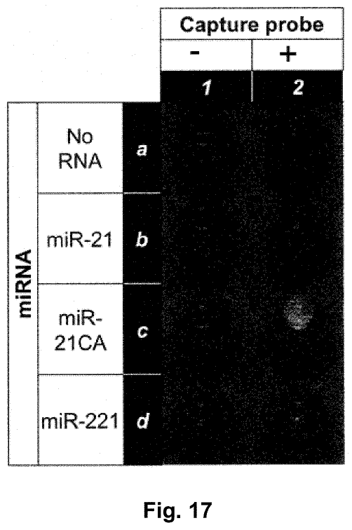

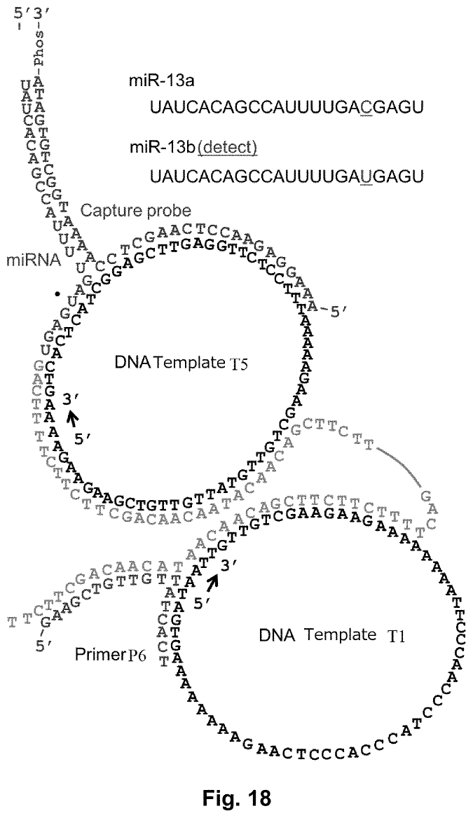

For detection of a single-base substitution of miRNA, the following experiment was carried out. FIG. 18 shows the reaction scheme. As shown in Table 9, there is a c/u single-base substitution between miR-13a and miR-13b.

For detection of this mutation, the following reagents were prepared, and extension reaction was carried out by incubation at 37° C. for 2 hours. To 8 μL of each reaction solution, 2 μL of 5×PBS153NM buffer (50 mM HP0₄²⁻, 730 mM Cl⁻, 765 mM Nat, 13.5 mM K⁺, 12.5 mM Mg²⁺; pH 7.4) was added. With 10 μL of the mixed solution, 2 μL of a fluorescent dye (30 μM ThT derivative (THT-HE, JP 2016-079132 A) solution in 1×PBS153NM buffer; final concentration (5 μM)) was mixed, and then the resulting mixture was incubated at 25° C. for 30 minutes. The prepared solution was subjected to irradiation using a 410-nm UV lamp. A photograph was taken with a camera equipped with a cut-off filter (which cuts off wavelengths shorter than 460 nm).

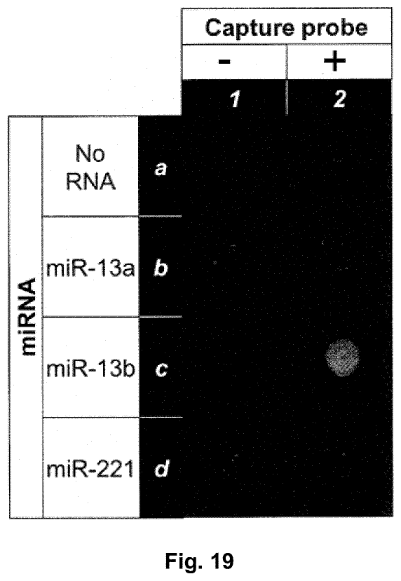

### Preparation of FIGS. 19 a1and a2

A mixture was prepared with 2 μL of 100 nM DNA template T5 (final concentration, 10 nM), 2 μL of 400 nM DNA template T1 (final concentration, 40 nM), 2 μL of water or 120 nM capture probe (2) (final concentration, 12 nM), 2 μL of 480 nM DNA primer P6 (final concentration, 48 nM), 2 μL of 10 x attached buffer, 2 μL of 10×attached BSA solution, 2 μL of 10 mM dNTPs (final concentration, 1 mM), 2 μL of 1 U/μL Phi29 Polymerase (final concentration, 0.1 U/μL), and 4 μL of water (20 μL in total).

### Preparation of FIG. 19 b1 and b2

A mixture was prepared with 2 μL of 100 nM DNA template T5 (final concentration, 10 nM), 2 μL of 400 nM DNA template T1 (final concentration, 40 nM), 2 μL of water or 120 nM capture probe (2) (final concentration, 12 nM), 2 μL of 480 nM DNA primer P6 (final concentration, 48 nM), 2 μL of 10 x attached buffer, 2 μL of 10×attached BSA solution, 2 μL of 10 mM dNTPs (final concentration, 1 mM), 2 μL of 1 U/μL Phi29 Polymerase (final concentration, 0.1 U/μL), 2 μL of 10 nM miR-13a (final concentration, 1 nM), and 2 μL of water (20 μL in total).

### Preparation of FIGS. 19 c1 and c2

A mixture was prepared with 2 μL of 100 nM DNA template T5 (final concentration, 10 nM), 2 μL of 400 nM DNA template T1 (final concentration, 40 nM), 2 μL of water or 120 nM capture probe (2) (final concentration, 12 nM), 2 μL of 480 nM DNA primer P6 (final concentration, 48 nM), 2 μL of 10 ×attached buffer, 2 μL of 10×attached BSA solution, 2 μL of 10 mM dNTPs (final concentration, 1 mM), 2 μL of 1 U/μL Phi29 Polymerase (final concentration, 0.1 U/μL), 2 μL of 10 nM miR-13b (final concentration, 1 nM), and 2 μL of water (20 μL in total).

### Preparation of FIGS. 19 d1 and d2

A mixture was prepared with 2 μL of 100 nM DNA template T5 (final concentration, 10 nM), 2 μL of 400 nM DNA template T1 (final concentration, 40 nM), 2 μL of water or 120 nM capture probe (2) (final concentration, 12 nM), 2 μL of 480 nM DNA primer P6 (final concentration, 48 nM), 2 μL of 10 x attached buffer, 2 μL of 10×attached BSA solution, 2 μL of 10 mM dNTPs (final concentration, 1 mM), 2 μL of 1 U/μL Phi29 Polymerase (final concentration, 0.1 U/μL), 2 μL of 10 nM miR-221 (final concentration, 1 nM), and 2 μL of water (20 μL in total).

TABLE 9
Sequences of oligonucleotides

| | Length | | |
|---|---|---|---|
| | (mer) | Sequence (5′→3′) | Modification |
| DNA Primer P6 | 18 | GAAGCTGTTGTTATCACT (SEQ ID NO: 41) | |
| Capture Probe (1) | 34 | AAAGGAGAACCTCAAGCTCCTCAGTCTGATAAGC | 3′ -end |
| | | (SEQ ID NO: 42) | phosphorylation |
| Capture Probe (2) | 35 | AAAGGAGAACCTCAAGCTCCAAAATGGCTGTGATA | 3′ -end |
| | | (SEQ ID NO: 43) | phosphorylation |
| DNA Template T4 | 67 | CTGTCAACAGGAGCTTGAGGTTCTCCTTTAAAAAGAAGCT | Circulated |
| (First single strand: | | GTTGTATTGTTGTCGAAGAAGAAAAGT | |
| circular DNA: miR-21) | | (SEQ ID NO: 44) | |
| DNA Template T1 | 62 | CCCAACCCTACCCACCCTCAAGAAAAAAAAGTGATAATTG | Circulated |
| (Second single strand | | TTGTCGAAGAAGAAAAAAAATT | |
| circular DNA) | | (SEQ ID NO: 38) | |
| DNA Template T5 | 66 | CACTCATCGGAGCTTGAGGTTCTCCTTTAAAAAGAAGCTG | Circulated |
| (First single strand | | TTGTATTGTTGTCGAAGAAGAAAAGT | |
| circular DNA: miR-13) | | (SEQ ID NO: 45) | |
| miR-21CA | 24 | uagcuuaucagacugauguugaca (SEQ ID NO: 46) | |
| miR-21 | 22 | uagcuuaucagacugauguuga (SEQ ID NO: 47) | |
| miR-13a | 22 | uaucacagccauuuugacgagu (SEQ ID NO: 48) | |
| miR-13b | 22 | uaucacagccauuuugaugagu (SEQ ID NO: 49) | |
| miR-221 | 23 | agcuacauugucugcuggguuuc (SEQ ID NO: 50) | |

Capital letter: DNA, Small letter: RNA

Each double-stranded DNA was prepared by annealing with the complementary strand.

### Results

### [1] Detection of Mutant miRNAs Having Different Chain Lengths (Example 6)

The present method is a method for detecting mutations causing differences in the chain length of miRNA. In the designing of the template, thermodynamics in the portion of hybridization between the target RNA and the template is taken into account. A past study has shown that the reaction proceeds in cases where −ΔG° of the hybridized portion exceeds 4.2 kcal/mol. Taking this fact into account, miR-21CA, which is a mutant, was designed to have a −ΔG° of 5.7 kcal/mol, and miR-21, which does not have a mutation, was designed to have a −ΔG° of 2.7 kcal/mol (FIG. 16).

In the actual experimental results, the reaction proceeded when the target was miR-21CA (FIG. 17 c2), but no progress of the reaction was found when the target was miR-21 (FIG. 17 b2).

Also in the cases where no capture probe was used (FIG. 17 a1, b1, c1, d1), and the case where a different target was used (FIG. 17 d2), no progress of the reaction was found.

### [2] Detection of miRNA Having Single Base Mutation (Example 7)

This method is a method for detecting a one-base difference of miRNA. In the designing of the template, thermodynamic stability was taken into account such that the template and the target miR-13b are complementary to each other (FIG. 18).

As a result of the experiment, in the case of miR-13a (FIG. 19 b2), the mismatch with the template did not allow the reaction to proceed. On the other hand, miR-13b (FIG. 19 c2) allowed the reaction to proceed because of the complete complementarity.

Also in the cases where no capture probe was used (FIG. 19 a1, b1, c1, d1), and the case where a different target was used (FIG. 19 d2), no progress of the reaction was found.

### [Example 8] Detection of Single Nucleotide Polymorphism in Long-chain DNA

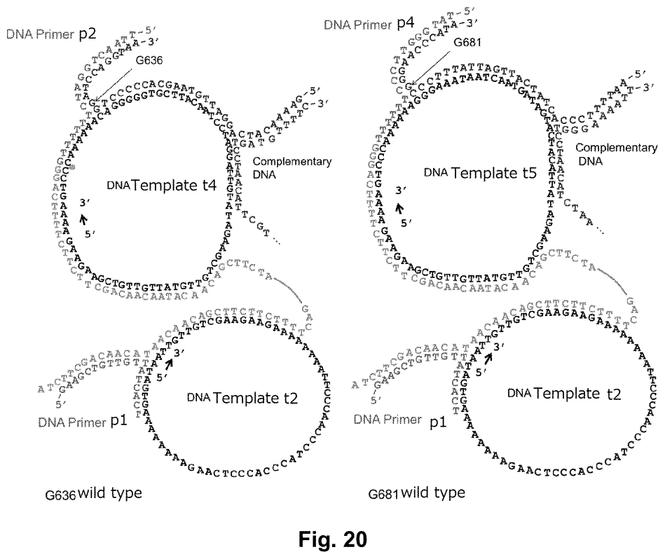

For detection of single nucleotide polymorphisms in a long-chain DNA, the reaction shown in FIG. 20 was carried out to detect the polymorphisms G636 and G681 contained in the long-chain DNA.

### 1. Preparation of Solution

### [1] Detection of Single Nucleotide Polymorphisms in Long-chain DNA (Use of PCR-amplified Sample)

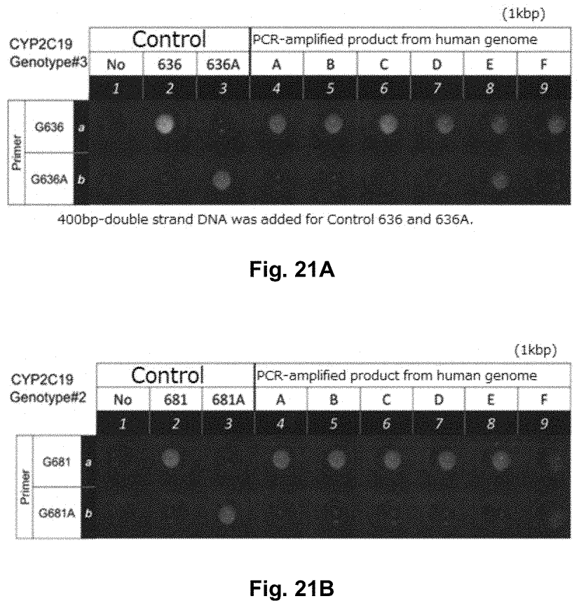

### Preparation of FIG. 21(A) a1to a9

A mixture was prepared with 2 μL of 100 nM DNA template t4 (final concentration, 10 nM), 2 μL of 400 nM DNA template t2 (final concentration, 40 nM), 2 μL of 120 nM primer p2 (final concentration, 12 nM), 2 μL of 480 nM DNA primer p1 (final concentration, 48 nM), 2 μL of 10 x attached buffer, 2 μL of 10×attached BSA solution, 2 μL of 10 mM dNTPs (final concentration, 1 mM), 2 μL of 1 U/μL Phi29 Polymerase (final concentration, 0.1 U/μL), 2 μL of water or 10 nM target DNA (40mer; annealing of double-stranded DNA was carried out in advance; final concentration, 1 nM), and 2 μL of water (20 μL in total).

### Preparation of FIG. 21(A) b1 to b9

A mixture was prepared with 2 μL of 100 nM DNA template t4 (final concentration, 10 nM), 2 μL of 400 nM DNA template t2 (final concentration, 40 nM), 2 μL of 120 nM primer p3 (final concentration, 12 nM), 2 μL of 480 nM DNA primer p1 (final concentration, 48 nM), 2 μL of 10×attached buffer, 2 μL of 10×attached BSA solution, 2 μL of 10 mM dNTPs (final concentration, 1 mM), 2 μL of 1 U/μL Phi29 Polymerase (final concentration, 0.1 U/μL), 2 μL of water or 10 nM target DNA (40mer; annealing of double-stranded DNA was carried out in advance; final concentration, 1 nM), and 2 μL of water (20 μL in total).

### Preparation of FIG. 21(B) a1 to a9

A mixture was prepared with 2 μL of 100 nM DNA template t5 (final concentration, 10 nM), 2 μL of 400 nM DNA template t2 (final concentration, 40 nM), 2 μL of 120 nM primer p4 (final concentration, 12 nM), 2 μL of 480 nM DNA primer p1 (final concentration, 48 nM), 2 μL of 10×attached buffer, 2 μL of 10 x attached BSA solution, 2 μL of 10 mM dNTPs (final concentration, 1 mM), 2 μL of 1 U/μL Phi29 Polymerase (final concentration, 0.1 U/μL), 2 μL of water or 10 nM target DNA (40mer; annealing of double-stranded DNA was carried out in advance; final concentration, 1 nM), and 2 μL of water (20 μL in total).

### Preparation of FIGS. 21(B) b1 to b9

A mixture was prepared with 2 μL of 100 nM DNA template t5 (final concentration, 10 nM), 2 μL of 400 nM DNA template t2 (final concentration, 40 nM), 2 μL of 120 nM primer p5 (final concentration, 12 nM), 2 μL of 480 nM DNA primer p1 (final concentration, 48 nM), 2 μL of 10 x attached buffer, 2 μL of 10 x attached BSA solution, 2 μL of 10 mM dNTPs (final concentration, 1 mM), 2 μL of 1 U/μL Phi29 Polymerase (final concentration, 0.1 U/μL), 2 μL of water or 10 nM target DNA (40mer; annealing of double-stranded DNA was carried out in advance; final concentration, 1 nM), and 2 μL of water (20 μL in total).

### [2] Detection of Single Nucleotide Polymorphism in Long-chain DNA (Use of Extract)

### (1) Preparation of Cell Extract (Total DNA)

Oral mucosa was taken by rubbing with a swab, and suspended in 150 μL of ISOHAIR EASY (manufactured by Nippon Gene). The sample was then treated according to the attached protocol. Thereafter, replacement with 20 mM Tris-HCl, pH 7.4 was carried out using a spin column (centrifugal ultrafiltration filter unit).

### [2] Preparation of Detection Samples

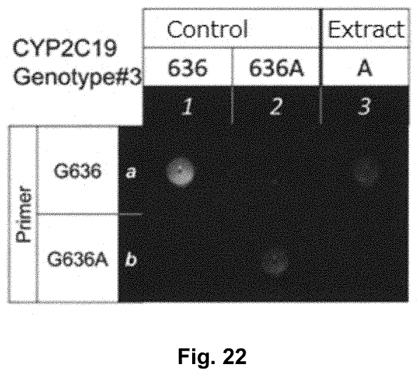

### Preparation of FIG. 22(A) a1 to a3

A mixture was prepared with 2 μL of 100 nM DNA template t4 (final concentration, 10 nM), 2 μL of 400 nM DNA template t2 (final concentration, 40 nM), 2 μL of 120 nM primer p2 (final concentration, 12 nM), 2 μL of 480 nM DNA primer p1 (final concentration, 48 nM), 2 μL of 10×attached buffer, 2 μL of 10×attached BSA solution, 2 μL of 10 mM dNTPs (final concentration, 1 mM), 2 μL of 1 U/μL Phi29 Polymerase (final concentration, 0.1 U/μL), 2 μL of water or 10 nM target DNA (40mer; annealing of double-stranded DNA was carried out in advance; final concentration, 1 nM), and 2 μL of water (20 μL in total).

Preparation of FIG. 22(A) b1 to b3

A mixture was prepared with 2 μL of 100 nM DNA template p4 (final concentration, 10 nM), 2 μL of 400 nM DNA template t2 (final concentration, 40 nM), 2 μL of 120 nM primer p3 (final concentration, 12 nM), 2 μL of 480 nM DNA primer p1 (final concentration, 48 nM), 2 μL of 10×attached buffer, 2 μL of 10×attached BSA solution, 2 μL of 10 mM dNTPs (final concentration, 1 mM), 2 μL of 1 U/μL Phi29 Polymerase (final concentration, 0.1 U/μL), 2 μL of water or 10 nM target DNA (40mer; annealing of double-stranded DNA was carried out in advance; final concentration, 1 nM), and 2 μL of water (20 μL in total).

TABLE 10
Sequences of oligonucleotides

| | Length | |
|---|---|---|
| | (mer) | Sequence (5′→3′) |
| DNA Primer | 18 | GAAGCTGTTGTTATCACT |
| P1 | | (SEQ ID NO: 33) |
| DNA Primer | 18 | TTACCTGGATTTTTTTTG |
| p2 | | (SEQ ID NO: 34) |
| DNA Primer | 18 | TTACCTGGATTTTTTTTG |
| P3 | | (SEQ ID NO: 35) |
| DNA Primer | 18 | TATGGGTTCCCTTTTTTG |
| P4 | | (SEQ ID NO: 36) |
| DNA Primer | 18 | TATGGGTTCCTTTTTTTG |
| P5 | | (SEQ ID NO: 37) |
| Positive | 30 | AGCTCCTTTTTTGGGACTTTTCTTCTTCGA |
| control | | (SEQ ID NO: 51) |
| DNA Template | 62 | CCCAACCGTACCCACCCTCAAGAAAAAAAAGTGATAATTGTTGTCGAAGAAG |
| t2 | | (SEQ ID NO: 52) |
| DNA Template | 73 | CAAAAAACAGGGGGTGCTTACAATCCTAGGATTGTATAGAAGCTGTTGTATTGTTGTCGAAGAAGAA |
| t4 | | AAGTCC (SEQ ID NO: 39) |
| DNA Template | 73 | CAAAAAAGGGAAATAATCAATGATAGTACTATCATTTAGAAGCTGTTGTATTGTTGTCGAAGAAGAA |
| t5 | | AAGTCC (SEQ ID NO: 40) |
| G636 | 40 | GAAAACATCAGGATTGTAAGCACCCCCCTGGATCCAGGTAA (SEQ ID NO: 53) |
| G636 | 40 | TTACCTGGATCCAGGGGGTGCTTACAATCCTGATGTTTTC (SEQ ID NO: 54) |
| Compementary | | |
| G636A | 40 | GAAAACATCAGGATTGTAAGCACCCCCTGAATCCAGGTAA (SEQ ID NO: 55) |
| G636A | 40 | TTACCTGGATTCAGGGGGTGCTTACAATCCTGATGTTTTC (SEQ ID NO: 56) |
| Compementary | | |
| G681 | 40 | AATTTTCCCACTATCATTGATTATTTCCCGGGAACCCATA (SEQ ID NO: 57) |
| G681 | 40 | TATGGGTTCCCGGGAAATAATCAATGATAGTGGGAAAATT (SEQ ID NO: 58) |
| Compementary | | |
| G681A | 40 | AATTTTCCCACTATCATTGATTATTTCCCAGGAACCCATA (SEQ ID NO: 59) |
| G681A | 40 | TATGGGTTCCTGGGAAATAATCAATGATAGTGGGAAAATT (SEQ ID NO: 60) |
| Compementary | | |
| G636 1 kbp | 1k | Amplified sample including the mutation site together with 500 bp sequences |
| | | before and after the site |
| G681 1 kbp | 1k | Amplified sampie including the mutation site together with 500 bp sequences |
| | | before and after the site. |

Each double-stranded DNA was prepared by annealing with the complementary strand.

The sample of long-chain DNA of 1 kbp containing G636 and G681 was prepared by PCR amplification of the gene from human oral mucosa.

Reagents Used:
ThT (thioflavin T) derivative
5×PBS153NM
50 mM HPO₄²⁻, 730 mM Cl⁻765 mM Na+, 13.5 mM K⁺, 12.5 mM Mg²+; pH 7.4
1×PBS153NM
10 mM HPOhd 4²⁻, 146 mM Cl⁻, 153 mM Na⁺, 2.7 mM K⁺, 2.5 mM Mg²⁺; pH 7.4
ISOHAIR EASY (manufactured by Nippon Gene)

### 2. Polymerase Reaction

The solution prepared in 1. was incubated at 37° C. for 2 hours.

### 3. Visual Detection

To 8 μL of the reaction solution of 2., 2 μL of 5×PBS153NM buffer was added.

With 10 μL of the mixed solution, 2 μL of a fluorescent dye (30 μM ThT derivative solution in 1×PBS153NM buffer; final concentration (5 μM)) was mixed, and then the resulting mixture was incubated at 25° C. for 30 minutes.

The prepared solution was subjected to irradiation using a 410-nm UV lamp. A photograph was taken with a camera equipped with a cut-off filter (which cuts off wavelengths shorter than 460 nm).

### Results

### [1] Detection of Single Nucleotide Polymorphisms in Long-chain DNA (Use of PCR-amplified Sample)

This method is a method for detecting single nucleotide polymorphisms (SNPs) in the genome. In FIG. 21(A), a mutation (mutation from G to A) of the base at position 636 (G681, Genotype #3) in the CYP2C19 gene, which encodes cytochrome P450, is detected. In FIG. 21(B), a mutation (mutation from G to A) of the base at position 681 (G636, Genotype #2) in the CYP2C19 gene is detected. Primers corresponding to the wild type and the mutant type, respectively, were used. (FIG. 20)

The genomic sample of human oral mucosal cells was prepared by PCR amplification of a 1-kbp region (500 bp each of the sequences before and after the mutation site). The analysis was carried out for six individuals. As a result, G636 showed the heterozygote (mixture of the wild type and the mutant type) in E, and G681 showed the heterozygote in F.

Thus, mutations can be confirmed even in cases where the target is a long-chain double-stranded DNA of 1 kbp.

### [2] Detection of Single Nucleotide Polymorphism in Long-chain DNA (Use of Extract)

This method is a method for detecting single nucleotide polymorphisms (SNPs) in a cell extract (in total genome). In FIG. 22, a mutation (mutation from G to A) of the base at position 636 (G681, Genotype #3) in the CYP2C19 gene, which encodes cytochrome P450, is detected. Primers corresponding to the wild type and the mutant type, respectively, were used. (FIG. 20)

As a result, the one base could be identified even in the case where the total genome was used (FIG. 22).

### DESCRIPTION OF SYMBOLS

10 . . . Single-stranded circular DNA; 11 . . . target polynucleotide; 12 . . . oligonucleotide primer; 13 . . . amplification product (extension product); 101 . . . sequence complementary to the first region; 102 . . . primer-binding sequence; 103 . . . sequence complementary to the guanine quadruplex-forming sequence; 104 . . . sequence containing the guanine quadruplex; 105 . . . guanine quadruplex detection reagent; 111 . . . first region; 112 . . . second region; 121 . . . sequence complementary to the second region; 122 . . . sequence complementary to the primer-binding region.

20 . . . Single-stranded circular DNA; 21 . . . target polynucleotide; 22 . . . first oligonucleotide primer; 23 . . . first amplification product (extension product); 24 . . . second single-stranded circular DNA; 25 . . . second oligonucleotide primer; 26 . . . second amplification product (extension product); 201 . . . sequence complementary to the first region; 202 . . . first primer-binding sequence; 203 . . . sequence complementary to the second-single-stranded-circular-DNA-binding sequence; 204 . . . region adjacent to the 5′-side of 203; 211 . . . first region; 212 . . . second region; 221 . . . sequence complementary to the second region; 222 . . . sequence complementary to the first primer-binding region; 231 . . . region complementary to 203; 232 . . . region complementary to the region 204; 241 . . . same sequence as the sequence 203 complementary to the second-single-stranded-circular-DNA-binding sequence; 242 . . . second primer-binding sequence; 243 . . . sequence complementary to the guanine quadruplex-forming sequence; 251 . . . same sequence as the region 204; 252 . . . sequence complementary to the second primer-binding sequence 242 of the second single-stranded circular DNA; 261 . . . sequence containing the guanine quadruplex; 262 . . . guanine quadruplex detection reagent.

30 . . . Single-stranded circular DNA; 31 . . . capture polynucleotide; 32 . . . miRNA; 33 . . . amplification product (extension product); 34 . . . guanine quadruplex detection reagent; 301 . . . sequence complementary to the second region of the miRNA; 302 . . . second region of the single-stranded circular DNA; 303 . . . sequence complementary to the guanine quadruplex-forming sequence; 311 . . . sequence complementary to the second region of the single-stranded circular DNA; 312 . . . sequence complementary to the first region of the miRNA; 321 . . . first region of the miRNA; 322 . . . second region containing the mutation of the miRNA; 331 . . . guanine quadruplex-forming sequence.

40 . . . Single-stranded circular DNA; 41 . . . capture polynucleotide; 42 . . . miRNA; 43 . . . first amplification product (extension product); 44 . . . second single-stranded circular DNA; 45 . . . second oligonucleotide primer; 46 . . . second amplification product (extension product); 47 . . . guanine quadruplex detection reagent; 401 . . . sequence complementary to the second region of the miRNA; 402 . . . second region of the single-stranded circular DNA; 403 . . . sequence complementary to the second-single-stranded-circular-DNA-binding sequence; 404 . . . region adjacent to the 5′-side of 403; 411 . . . sequence complementary to the second region of the single-stranded circular DNA; 412 . . . sequence complementary to the first region of the miRNA; 421 . . . first region of the miRNA; 422 . . . second region containing the mutation of the miRNA; 431 . . . region complementary to 403; 432 . . . region complementary to the region 404; 433 . . . sequence complementary to the sequence 403; 441 . . . same sequence as the sequence 403 complementary to the second-single-stranded-circular-DNA-binding sequence; 442 . . . second primer-binding sequence; 443 . . . sequence complementary to the guanine quadruplex-forming sequence; 451 . . . same sequence as the region 404; 452 . . . sequence complementary to the second primer-binding sequence 442 of the second single-stranded circular DNA; 461 . . . guanine quadruplex-forming sequence.

1. A method for detecting a gene mutation(s), the method comprising the steps of:
hybridizing a single-stranded circular DNA and an oligonucleotide primer with a capture polynucleotide containing: a first region; and a second region adjacent to the 3′-side of the first region;
performing a nucleic acid amplification reaction by rolling circle amplification based on formation of a complex of the capture polynucleotide, the primer, and the single-stranded circular DNA; and
detecting amplified nucleic acid with a detection reagent;
wherein
the single-stranded circular DNA contains:
a sequence complementary to the first region of the capture polynucleotide; and
a primer-binding sequence adjacent to the 5′-side thereof;
the oligonucleotide primer contains:
a region having a sequence complementary to the second region of the capture polynucleotide; and
a region adjacent to the 3′-side thereof and having a sequence complementary to the primer-binding sequence of the single-stranded circular DNA; and
the capture polynucleotide or the oligonucleotide primer contains a mutation, and mutation-specific hybridization of the capture polynucleotide, the primer, and the single-stranded circular DNA occurs to form the ternary complex to cause the nucleic acid amplification reaction, followed by the detection with the detection reagent.

2. A method for detecting a gene mutation(s), the method comprising the steps of:
hybridizing a single-stranded circular DNA and a primer with a target polynucleotide containing: a first region; and a second region adjacent to the 3′-side of the first region and containing a mutation;
performing a nucleic acid amplification reaction by rolling circle amplification based on formation of a complex of the target polynucleotide, the primer, and the single-stranded circular DNA; and
detecting amplified nucleic acid with a detection reagent;
wherein
the single-stranded circular DNA contains:
a sequence complementary to the first region of the target polynucleotide; and
a primer-binding sequence adjacent to the 5′-side thereof; and
the oligonucleotide primer contains:
a region having a sequence complementary to the second region of the target polynucleotide; and
a region adjacent to the 3′-side thereof and having a sequence complementary to the primer-binding sequence of the single-stranded circular DNA.

3. The method for detecting a gene mutation(s) according to claim 1, wherein the single-stranded circular DNA further contains a sequence complementary to a detection reagent-binding sequence.

4. A method for detecting a gene mutation(s), the method comprising the steps of:
hybridizing a first single-stranded circular DNA and a first primer with a target polynucleotide containing: a first region; and a second region adjacent to the 3′-side of the first region and containing a mutation;
performing a nucleic acid amplification reaction by rolling circle amplification based on formation of a complex of the target polynucleotide, the first primer, and the first single-stranded circular DNA;
hybridizing a second single-stranded circular DNA and a second oligonucleotide primer with an extended chain generated by the nucleic acid amplification reaction, and performing a nucleic acid amplification reaction based on formation of a complex of the extended chain, the second primer, and the second single-stranded circular DNA; and
detecting amplified nucleic acid with a detection reagent;
wherein
the first single-stranded circular DNA contains:
a sequence complementary to the first region of the target polynucleotide;
a primer-binding sequence adjacent to the 5′-side thereof; and
a sequence complementary to a second-single-stranded-circular-DNA-binding sequence;
the first oligonucleotide primer contains:
a region having a sequence complementary to the second region of the target polynucleotide; and
a region adjacent to the 3′-side thereof and having a sequence complementary to the primer-binding sequence of the first single-stranded circular DNA;
the second single-stranded circular DNA contains:
the same sequence as the sequence, in the first single-stranded circular DNA, complementary to the second-single-stranded-circular-DNA-binding sequence; and
a second primer-binding sequence adjacent to the 5′-side thereof; and
the second oligonucleotide primer contains:
the same sequence as the region adjacent to the 5′-side of the sequence, in the first single-stranded circular DNA, complementary to the second-single-stranded-circular-DNA-binding sequence; and
a sequence adjacent to the 3′-side thereof and complementary to the second primer-binding sequence in the second single-stranded circular DNA.

5. The method for detecting a gene mutation(s) according to claim 4, wherein the second single-stranded circular DNA further contains a sequence complementary to a detection reagent-binding sequence.

6. The method for detecting a gene mutation(s) according to claim 2, wherein the oligonucleotide primer has a base that hybridizes with a mutated base present in the second region of the target polynucleotide, the base in the primer being located at the 3′-end of a region having a sequence of 8 to 15 bases complementary to the second region of the target polynucleotide.

7. The method for detecting a gene mutation(s) according to claim 4, wherein the first oligonucleotide primer has a base that hybridizes with a mutated base present in the second region of the target polynucleotide, the base in the primer being located at the 3′-end of a region having a sequence of 8 to 15 bases complementary to the second region of the target polynucleotide.

8. A method for detecting a gene mutation(s), the method comprising the steps of:
hybridizing a single-stranded circular DNA and a capture polynucleotide with a miRNA containing: a first region; and a second region located in the 3′-side thereof and containing a mutation;
performing a nucleic acid amplification reaction by rolling circle amplification based on formation of a complex of the capture polynucleotide, the miRNA, and the single-stranded circular DNA; and
detecting amplified nucleic acid with a detection reagent;
wherein
the single-stranded circular DNA contains:
a miRNA-binding region complementary to the second region of the miRNA; and
a second region in the 3′-side thereof; and
the capture polynucleotide contains:
a sequence complementary to the second region of the single-stranded circular DNA; and
a miRNA-binding sequence complementary to the first region of the miRNA.

9. The method for detecting a gene mutation(s) according to claim 8, wherein the single-stranded circular DNA further contains a sequence complementary to a detection reagent-binding sequence.

10. A method for detecting a gene mutation(s), the method comprising the steps of:
hybridizing a first single-stranded circular DNA and a capture polynucleotide with a miRNA containing: a first region; and a second region adjacent to the 3′-side of the first region and containing a mutation;
performing a nucleic acid amplification reaction by rolling circle amplification based on formation of a complex of the miRNA, the capture polynucleotide, and the first single-stranded circular DNA;
hybridizing a second single-stranded circular DNA and a second oligonucleotide primer with an extended chain generated by the nucleic acid amplification reaction, and performing a nucleic acid amplification reaction based on formation of a complex of the extended chain, the second primer, and the second single-stranded circular DNA; and
detecting amplified nucleic acid with a detection reagent;
wherein
the first single-stranded circular DNA contains:
a miRNA-binding region complementary to the second region of the miRNA;
a second region in the 3′-side thereof; and
a sequence complementary to a second-single-stranded-circular-DNA-binding sequence;
the capture polynucleotide contains:
a sequence complementary to the second region of the single-stranded circular DNA; and
a sequence complementary to the first region of the miRNA;
the second single-stranded circular DNA contains:
the same sequence as the sequence, in the first single-stranded circular DNA, complementary to the second-single-stranded-circular-DNA-binding sequence; and
a second primer-binding sequence adjacent to the 5′ side thereof; and
the second oligonucleotide primer contains:
the same sequence as the region adjacent to the 5′-side of the sequence, in the first single-stranded circular DNA, complementary to the second-single-stranded-circular-DNA-binding sequence; and
a sequence adjacent to the 3′-side thereof and complementary to the second primer-binding sequence in the second single-stranded circular DNA.

11. The method for detecting a gene mutation(s) according to claim 10, wherein the second single-stranded circular DNA further contains a sequence complementary to a detection reagent-binding sequence.

12. The method for detecting a gene mutation(s) according to claim 3, wherein the detection reagent-binding sequence is a guanine quadruplex-forming sequence, and the detection reagent is a guanine quadruplex-binding reagent.

13. The method for detecting a gene mutation(s) according to claim 12, wherein the sequence complementary to the guanine quadruplex-forming sequence contains a C₃N₁₋₁₀C₃N₁₋₁₀C₃N₁₋₁₀C₃ sequence.

14. The method for detecting a gene mutation(s) according to claim 12,wherein the guanine quadruplex-binding reagent contains a compound represented by the following General Formula (I): 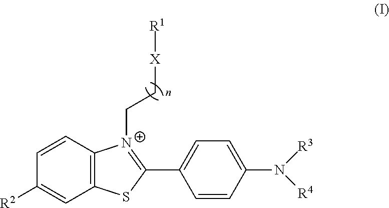
wherein
R¹ represents hydrogen, or a hydrocarbon group which optionally contains one or more selected from the group consisting of O, S, and N;
R², R³, and R⁴ each independently represent a C₁-C₅ hydrocarbon group;
n represents an integer of 0 to 5; and
X represents O, S, or NH.

15. The method for detecting a gene mutation(s) according to claim 1, wherein the gene mutation(s) is/are a single nucleotide polymorphism(s).

16. The method for detecting a gene mutation(s) according to claim 2, wherein the single-stranded circular DNA further contains a sequence complementary to a detection reagent-binding sequence.

17. The method for detecting a gene mutation(s) according to claim 5, wherein the detection reagent-binding sequence is a guanine quadruplex-forming sequence, and the detection reagent is a guanine quadruplex-binding reagent.

18. The method for detecting a gene mutation(s) according to claim 9, wherein the detection reagent-binding sequence is a guanine quadruplex-forming sequence, and the detection reagent is a guanine quadruplex-binding reagent.

19. The method for detecting a gene mutation(s) according to claim 11, wherein the detection reagent-binding sequence is a guanine quadruplex-forming sequence, and the detection reagent is a guanine quadruplex-binding reagent.
